(19) United States
(12) Patent Application Publication
(10) Pub. No.: US 2024/0423537 A1
(43) Pub. Date: Dec. 26, 2024

(54) METABOLISM MEASUREMENT APPARATUS, METABOLISM CALCULATION METHOD, AND METABOLISM CALCULATION PROGRAM

(71) Applicant: HAMAMATSU PHOTONICS K.K., Hamamatsu-shi, Shizuoka (JP)

(72) Inventors: Tomoya NAKAZAWA, Hamamatsu-shi, Shizuoka (JP); Rui SEKINE, Hamamatsu-shi, Shizuoka (JP); Masato KITABYASHI, Hamamatsu-shi, Shizuoka (JP); Yu HASHIMOTO, Hamamatsu-shi, Shizuoka (JP); Anna IENAKA, Hamamatsu-shi, Shizuoka (JP)

(73) Assignee: HAMAMATSU PHOTONICS K.K., Hamamatsu-shi, Shizuoka (JP)

(21) Appl. No.: 18/705,020

(22) PCT Filed: Aug. 10, 2022
(86) PCT No.: PCT/JP2022/030669

§ 371 Date: Apr. 26, 2024

(30) Foreign Application Priority Data
Nov. 8, 2021 (JP) 2021-181576

(51) Int. Cl. A61B 5/00; A61B 5/145; A61B 5/1455

(52) U.S. Cl. CPC A61B 5/4866; A61B 5/14532; A61B 5/14551

(57) ABSTRACT

A metabolism measurement apparatus includes, a photodetector that detects measurement light propagating through the living body and generates a detection signal according to an intensity of the measurement light, and a calculator. The calculator calculates a first parameter and a second parameter based on the detection signal. Each of the first parameter and the second parameter is a temporal relative change amount depending on a concentration of each of O₂Hb and HHb in blood and a volume of a blood vessel in an optical path. The calculator obtains, based on the first parameter and the second parameter, a numerical value regarding a concentration of HHb in blood from which an influence of the volume of the blood vessel in the optical path is excluded. The calculator obtains data regarding a degree of metabolism based on the numerical value.

### TECHNICAL FIELD

The present disclosure relates to a metabolism measurement apparatus, a metabolism calculation method, and a metabolism calculation program.

### BACKGROUND ART

Patent Literature 1 discloses a blood glucose level measurement apparatus. The blood glucose level measurement apparatus includes a light output unit, a light detector, and a calculator. The light output unit outputs measurement light to be input to a living body. The light detector detects the measurement light propagating through the living body and generates a detection signal according to the intensity of the measurement light. The calculator obtains a time lag between a temporal change in a first parameter regarding a concentration of oxygenated hemoglobin and a temporal change in a second parameter regarding a concentration of deoxygenated hemoglobin based on the detection signal and obtains data regarding a blood glucose level based on the time lag.

### CITATION LIST

### Patent Literature

[Patent Literature 1] Japanese Unexamined Patent Publication No. 2018-57511

### SUMMARY OF INVENTION

### Technical Problem

In recent years, apparatuses and methods for noninvasively measuring metabolism of a body have been developed. For example, a method that measures a heat flow caused by metabolism of glucose in a body by a thermometer and estimates a blood glucose level from the heat flow is known. However, in such a method, because a certain amount of time is required for heat conduction from a part of the body to the thermometer, there is a problem in that a lot of time is required for measurement.

An object of the present disclosure is to provide a metabolism measurement apparatus, a metabolism calculation method, and a metabolism calculation program capable of measuring metabolism of a body noninvasively and in a short time.

### Solution to Problem

A metabolism measurement apparatus according to the present disclosure is an apparatus that measures a degree of metabolism of a living body, and includes a light output unit, a light detector, and a calculator. The light output unit outputs measurement light to be input to the living body. The light detector detects the measurement light propagating through the living body and generates a detection signal according to an intensity of the measurement light. The calculator outputs data regarding the degree of metabolism based on the detection signal. The calculator has a first calculation portion, a second calculation portion, and a third calculation portion. The first calculation portion obtains a first parameter and a second parameter based on the detection signal. The first parameter is a temporal relative change amount from a certain timing and depends on a concentration of oxygenated hemoglobin in blood and a volume of a blood vessel in an optical path. The second parameter is a temporal relative change amount from a certain timing and depends on a concentration of deoxygenated hemoglobin in blood and the volume of the blood vessel in the optical path. The second calculation portion obtains at least one parameter among a third parameter and a fourth parameter based on the first parameter and the second parameter. The third parameter is a parameter regarding a concentration of oxygenated hemoglobin in blood from which an influence of the volume of the blood vessel in the optical path is excluded. The fourth parameter is a parameter regarding a concentration of deoxygenated hemoglobin in blood from which an influence of the volume of the blood vessel in the optical path is excluded. The third calculation portion obtains the data regarding the degree of metabolism based on the at least one parameter among the third parameter and the fourth parameter.

A metabolism calculation method according to the present disclosure is a method that calculates a degree of metabolism of a living body, and includes a first calculation step, a second calculation step, and a third calculation step. In the first calculation step, a first parameter and a second parameter are obtained. The first parameter is a temporal relative change amount from a certain timing and depends on a concentration of oxygenated hemoglobin in blood and a volume of a blood vessel in an optical path, in the living body. The second parameter is a temporal relative change amount from a certain timing and depends on a concentration of deoxygenated hemoglobin in blood and the volume of the blood vessel in the optical path. In the second calculation step, at least one parameter among a third parameter and a fourth parameter is obtained based on the first parameter and the second parameter. The third parameter is a parameter regarding a concentration of oxygenated hemoglobin in blood from which an influence of the volume of the blood vessel in the optical path is excluded. The fourth parameter is a parameter regarding a concentration of deoxygenated hemoglobin in blood from which an influence of the volume of the blood vessel in the optical path is excluded. In the third calculation step, the data regarding the degree of metabolism is obtained based on the at least one parameter among the third parameter and the fourth parameter.

A metabolism calculation program according to the present disclosure is a program that calculates a degree of metabolism of a living body, and causes a computer to execute a first calculation step, a second calculation step, and a third calculation step. In the first calculation step, a first parameter and a second parameter are obtained. The first parameter is a temporal relative change amount from a certain timing and depends on a concentration of oxygenated hemoglobin in blood and a volume of a blood vessel in an optical path, in the living body. The second parameter is a temporal relative change amount from a certain timing and depends on a concentration of deoxygenated hemoglobin in blood and the volume of the blood vessel in the optical path. In the second calculation step, at least one parameter among a third parameter and a fourth parameter is obtained based on the first parameter and the second parameter. The third parameter is a parameter regarding a concentration of oxygenated hemoglobin in blood from which an influence of the volume of the blood vessel in the optical path is excluded. The fourth parameter is a parameter regarding a concentration of deoxygenated hemoglobin in blood from which an influence of the volume of the blood vessel in the optical path is excluded. In the third calculation step, data regarding the degree of metabolism is obtained based on the at least one parameter among the third parameter and the fourth parameter.

The degree of metabolism of the body has a correlation with the concentration of oxygenated hemoglobin and the concentration of deoxygenated hemoglobin in blood. Accordingly, the degree of metabolism of the body can be measured noninvasively and in a short time by measuring one or both the concentration of oxygenated hemoglobin and the concentration of deoxygenated hemoglobin in blood. As a method that measures the concentrations of hemoglobin, near-infrared spectroscopy (NIRS) is known. In this method, each concentration of hemoglobin is measured in a body tissue that is present in the optical path of the measurement light. However, each concentration of hemoglobin in blood itself is not obtained, and the obtained numerical value depends on each concentration of hemoglobin in blood and the volume of the blood vessel in the optical path. Accordingly, the numerical value is affected by fluctuation of the volume of the blood vessel in the optical path, and in particular, fluctuation of an inner diameter of the blood vessel due to the beat of the heart, and the metabolism of the body cannot be calculated using the numerical value as it is with high accuracy.

Therefore, in the apparatus, the method, and the program described above, first, the first parameter that is the temporal relative change amount depending on the concentration of oxygenated hemoglobin in blood and the volume of the blood vessel in the optical path and the second parameter that is the temporal relative change amount depending on the concentration of deoxygenated hemoglobin in blood and the volume of the blood vessel in the optical path are obtained in the first calculation portion or the first calculation step in a similar manner to the NIRS of the related art. Then, at least one parameter among the third parameter regarding the concentration of oxygenated hemoglobin in blood from which the influence of the volume of the blood vessel in the optical path is excluded and the fourth parameter regarding the concentration of deoxygenated hemoglobin in blood from which the influence of the volume of the blood vessel in the optical path is excluded is obtained based on the first parameter and the second parameter in the second calculation portion or the second calculation step. The present inventors have conducted studies and have found that the parameter regarding each concentration of hemoglobin in blood from which the influence of the volume of the blood vessel in the optical path is excluded is obtained by calculation based on the numerical value obtained by the NIRS in this way. The data regarding the degree of metabolism can be obtained based on at least one parameter, in the third calculation portion or the third calculation step. Accordingly, with the apparatus, the method, and the program described above, the degree of metabolism of the body can be measured noninvasively, with high accuracy, and in a short time to an extent equivalent to the NIRS of the related art. In the apparatus, the method, and the program described above, the volume of the blood vessel indicates a total internal volume of the blood vessel in the optical path.

In the metabolism measurement apparatus, the second calculation portion may obtain the at least one parameter assuming that the volume of the blood vessel in the optical path is constant. Similarly, in the second calculation step of the metabolism calculation method and the metabolism calculation program, the at least one parameter may be obtained assuming that the volume of the blood vessel in the optical path is constant. In this case, the degree of metabolism of the body can be simply measured.

The metabolism measurement apparatus may further include a fourth calculation portion that obtains a numerical value regarding a fluctuation component of the volume of the blood vessel in the optical path from the first parameter and the second parameter. Then, the third calculation portion may obtain the data regarding the degree of metabolism based further on the numerical value regarding the fluctuation component of the volume of the blood vessel in the optical path obtained by the fourth calculation portion. Similarly, the metabolism calculation method and the metabolism calculation program may further include, before the third calculation step, a fourth calculation step of obtaining a numerical value regarding a fluctuation component of the volume of the blood vessel in the optical path from the first parameter and the second parameter. Then, in the third calculation step, the data regarding the degree of metabolism may be obtained based further on the numerical value regarding the fluctuation component of the volume of the blood vessel in the optical path obtained in the fourth calculation step. In this way, the fluctuation component of the volume of the blood vessel in the optical path can be obtained by calculation based on the numerical value obtained by the NIRS. According to the studies of the present inventors, reduction in measurement accuracy due to the fluctuation of the volume of the blood vessel in the optical path can be suppressed by obtaining data regarding the degree of metabolism based further on the numerical value regarding the fluctuation component of the volume of the blood vessel in the optical path. The fluctuation of the volume of the blood vessel in the optical path can be caused by, for example, an increase or a decrease in a total amount of hemoglobin in a period from a certain measurement to next measurement, a change in the optical path due to attachment and detachment of the light output unit and the light detector, or a change in the volume of the blood vessel in the optical path due to a local change in a blood flow.

The metabolism measurement apparatus may further include a fourth calculation portion that extracts steady components of the first parameter and the second parameter. The third calculation portion may obtain the data regarding the degree of metabolism based further on the steady components of the first parameter and the second parameter obtained by the fourth calculation portion. Similarly, the metabolism calculation method and the metabolism calculation program may further include, before the third calculation step, a fourth calculation step of extracting steady components of the first parameter and the second parameter. In the third calculation step, the data regarding the degree of metabolism may be obtained based further on the steady components of the first parameter and the second parameter obtained in the fourth calculation step. The steady components of the first parameter and the second parameter have a correlation with the volume of the blood vessel in the optical path. Accordingly, reduction in measurement accuracy due to the fluctuation of the volume of the blood vessel in the optical path can be suppressed by obtaining data regarding the degree of metabolism based further on the steady components of the first parameter and the second parameter.

In the metabolism measurement apparatus, the second calculation portion may obtain the third parameter based on Formula (1) in obtaining the third parameter, and obtain the fourth parameter based on Formula (2) in obtaining the fourth parameter. Similarly, in the second calculation step of the metabolism calculation method and the metabolism measurement program, the third parameter may be obtained based on Formula (1) in obtaining the third parameter and the fourth parameter may be obtained based on Formula (2) in obtaining the fourth parameter. Here, SpO₂ is an oxygen saturation, N_{oxy}(t) is the first parameter, N_{deoxy}(t) is the second parameter, C_{oxy,AC} is the third parameter, C_{deoxy,AC} is the fourth parameter, V_{DC} is a steady component of the volume of the blood vessel in the optical path, and α is a constant.

$$\begin{matrix}[ Formula 1 ] & \\ - {SpO}_{2} \cdot {N}_{deoxy} ( t ) + ( 1 - {SpO}_{2} ) \cdot {N}_{oxy} ( t ) \approx \frac{{V}_{D C}}{α} \cdot {C}_{oxy , A C} & ( 1 )\end{matrix}$$

$$\begin{matrix}[ Formula 2 ] & \\ {SpO}_{2} \cdot {N}_{deoxy} ( t ) - ( 1 - {SpO}_{2} ) \cdot {N}_{oxy} ( t ) \approx \frac{{V}_{D C}}{α} \cdot {C}_{deoxy , A C} & ( 2 )\end{matrix}$$

With this, the degree of metabolism of the body can be calculated with higher accuracy.

In the metabolism measurement apparatus, the third calculation portion may further obtain a blood glucose level of the living body based on a pre-acquired relationship between the degree of metabolism and the blood glucose level. Similarly, in the third calculation step of the metabolism calculation method and the metabolism measurement program, a blood glucose level of the living body may be further obtained based on a pre-acquired relationship between the degree of metabolism and the blood glucose level. In this case, the blood glucose level can be measured noninvasively and in a short time.

In the metabolism measurement apparatus, the second calculation portion may extract a quantity of predetermined feature from the at least one parameter, and the third calculation portion may obtain the data regarding the degree of metabolism based on a pre-acquired relationship between the quantity of predetermined feature and the degree of metabolism. Similarly, in the second calculation step of the metabolism calculation method and the metabolism measurement program, a quantity of predetermined feature may be extracted from the at least one parameter, and in the third calculation step, the data regarding the degree of metabolism may be obtained based on a pre-acquired relationship between the quantity of predetermined feature and the degree of metabolism. In this case, data regarding the degree of metabolism can be obtained by simple calculation with high accuracy. In this case, the quantity of predetermined feature may be at least one value selected from a group consisting of a maximum value, a time average value, a peak-to-peak value, and a time integration value, of the at least one parameter.

### Advantageous Effects of Invention

With the metabolism measurement apparatus, the metabolism calculation method, and the metabolism calculation program according to the present disclosure, the metabolism of the body can be measured noninvasively and in a short time.

### BRIEF DESCRIPTION OF DRAWINGS

FIG. 1 is a conceptual diagram of a metabolism measurement apparatus according to an embodiment.

FIG. 2 is a conceptual diagram of a light measurement instrument according to the embodiment.

FIG. 3 is a block diagram illustrating a configuration example of the metabolism measurement apparatus.

FIG. 4 is a block diagram illustrating functions of a calculator that are implemented by a CPU.

FIG. 5 is a schematic view illustrating a volume of a blood vessel in an optical path. Part (a) of FIG. 5 illustrates a case where a density of a blood vessel in an optical path is small in a certain optical path of measurement light. Part (b) of FIG. 5 illustrates a case where a density of a blood vessel in an optical path having the same length as in Part (a) of FIG. 5 is greater than in Part (a) of FIG. 5.

FIG. 6 is a schematic view illustrating a volume of a blood vessel in an optical path. Part (a) of FIG. 6 illustrates a case where an optical path length of a measurement light is short. Part (b) of FIG. 6 illustrates a case where a density of a blood vessel is the same as in Part (a) of FIG. 6 and an optical path length of an optical path is longer than that in Part (a) of FIG. 6.

FIG. 7 is a schematic view illustrating a volume of a blood vessel in an optical path. Part (a) of FIG. 7 illustrates a case where a blood vessel is thin. Part (b) of FIG. 7 illustrates a case where a blood vessel is thicker than in Part (a) of FIG. 7.

Part (a) of FIG. 8 is a graph illustrating change over time in detected light intensity at a wavelength of 660 mm and change over time in detected light intensity at a wavelength of 910 mm. Part (b) of FIG. 8 is a graph illustrating change over time in Δoxy-Hb at the wavelength of 660 mm and change over time in Δdeoxy-Hb at the wavelength of 910 mm. Part (c) of FIG. 8 is a graph illustrating a result of executing filter processing of removing a low-frequency component smaller than a heartbeat frequency on the graph illustrated in Part (b) of FIG. 8.

Part (a) of FIG. 9 is a graph illustrating an example of change over time in a temporal relative change amount of a concentration of HHb. Part (b) of FIG. 9 is a graph illustrating an example of change over time in a value of a right side of Formula (29).

FIG. 10 is a flowchart illustrating a metabolism calculation method according to the embodiment.

Part (a) of FIG. 11 is a graph illustrating a time waveform of the temporal relative change amount of the concentration of HHb. Part (b) of FIG. 11 is a graph illustrating a time waveform of a temporal relative change amount of a concentration of O₂Hb corresponding to Part (a) of FIG. 11.

Part (a) to Part (c) of FIG. 12 are graphs illustrating measurement results of three subjects, respectively.

FIG. 13 is a graph illustrating a relationship between a metabolism index estimated by the metabolism measurement apparatus and an actual blood glucose level.

FIG. 14 is a diagram illustrating a graph G51 that is a sinusoidal time waveform generated in a simulative manner for examination, on which white noise is superimposed, a graph G52 obtained by subjecting an integration type filter to the graph G51, and a graph G53 obtained by subjecting a differentiation type filter to the graph G51.

Graphs G61 and G62 of Part (a) of FIG. 15 illustrate results of extracting maximum values of the respective graphs G51 and G52 of FIG. 14, respectively. Graphs G71 and G72 of Part (b) of FIG. 15 illustrate results of extracting peak-to-peak values of the respective graphs G51 and G52 of FIG. 14, respectively. Graphs G81 and G82 of Part (c) of FIG. 15 illustrate results of extracting time average values of the respective graphs G51 and G52 of FIG. 14, respectively.

FIG. 16 is a diagram illustrating a graph G91 that is a sinusoidal time waveform generated in a simulative manner for examination, on which low-frequency undulations are superimposed, a graph G92 obtained by applying an integration type filter to the graph G91, and a graph G93 obtained by applying a differentiation type filter to the graph G91.

Graphs G101 to G103 of Part (a) of FIG. 17 illustrate results of extracting maximum values of the respective graphs G91 to G93 of FIG. 16, respectively. Graphs G111 to G113 of Part (b) of FIG. 17 illustrate results of extracting peak-to-peak values of the respective graphs G91 to G93 of FIG. 16, respectively. Graphs G121 to G123 of Part (c) of FIG. 17 illustrate results of extracting time average values of the respective graphs G91 to G93 of FIG. 16, respectively.

### DESCRIPTION OF EMBODIMENTS

Hereinafter, an embodiment of a metabolism measurement apparatus, a metabolism calculation method, and a metabolism calculation program according to the present disclosure will be described in detail with reference to the accompanying drawings. In the description of the drawings, the same components are represented by the same reference numerals, and redundant description will not be repeated.

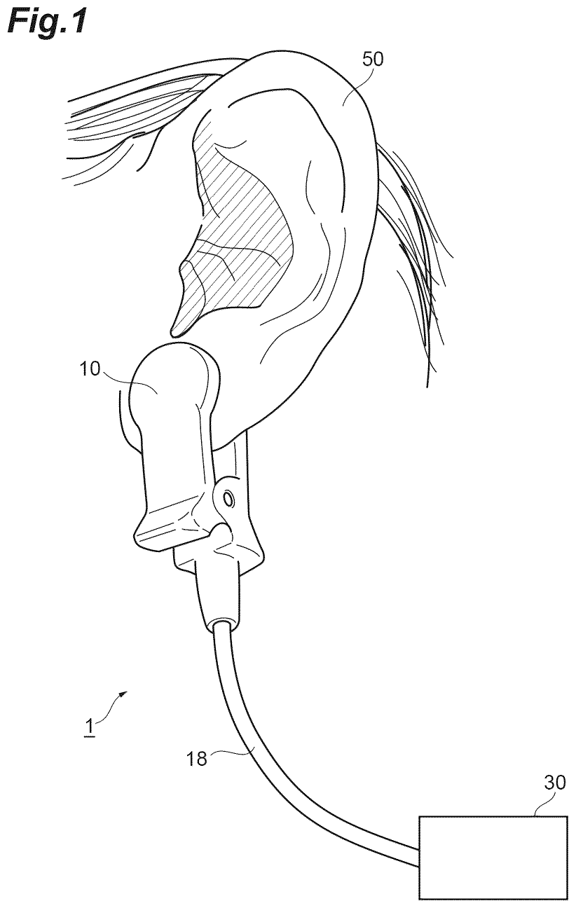

FIG. 1 is a conceptual diagram of a metabolism measurement apparatus 1 according to the present embodiment. The metabolism measurement apparatus 1 includes a light measurement instrument (probe) 10 and a main body unit 30. The main body unit 30 measures a degree of metabolism of a living body 50 based on the intensity of light detected from the living body 50 by the light measurement instrument 10. The light measurement instrument 10 may sandwich the living body 50 such as a finger or an ear or may be fixed to the living body 50 such as a head by an expandable band, for example. The main body unit 30 informs a subject of data regarding the measured degree of metabolism. The main body unit 30 can be configured with, for example, a computer. The computer is, for example, a smart device. The smart device is, for example, a smartphone or a tablet terminal. The light measurement instrument 10 is electrically connected to the main body unit 30 via a cable 18.

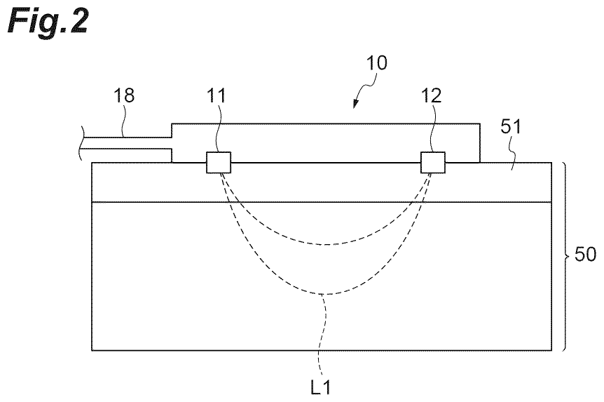

FIG. 2 is a conceptual diagram of the light measurement instrument 10 according to the present embodiment. The light measurement instrument 10 has a light source (light output unit) 11 and a photodetector (light detector) 12. The light source 11 is disposed at a predetermined light input position on a surface of a skin 51 of the living body 50 and outputs measurement light L1. A wavelength of the measurement light L1 is sequentially switched to N predetermined wavelengths (λ₁, λ₂, . . . , λ_{N}). N is an integer equal to or greater than 2. The measurement light L1 propagates through the living body 50 and is output from a predetermined light detection position on the surface of the skin 51 of the living body 50. The photodetector 12 detects the measurement light L1 output from the surface of the skin 51 of the living body 50 and generates a detection signal according to the intensity of the detected measurement light L1. The predetermined wavelengths are included in a range of, for example, a red wavelength region of visible light and a near infrared region, that is, 670 nm to 2500 nm. In an example, N=3, and λ₁, λ₂, and λ₃ are, for example, 735 nm, 810 nm, and 850 nm, respectively. When spectroscopic measurement can be performed in the photodetector 12, the light source 11 may output broad measurement light L1 having a wavelength range including the N predetermined wavelengths (λ₁, λ₂, . . . , and λ_{N}).

The light source 11 is, for example, a semiconductor light-emitting element such as a light-emitting diode (LED), a laser diode (LD), or a super-luminescent diode (SLD). The measurement light L1 output from the light source 11 is input substantially vertically with respect to the surface of the skin 51 of the living body 50. The photodetector 12 has a light detection element and a preamplifier. The photodetector 12 detects the measurement light L1 propagating through the living body 50 and generates the detection signal according to the intensity of the measurement light L1. The light detection element has light receiving sensitivity to a wavelength region including a center wavelength of the measurement light L1 output from the light source, for example. The light detection element is, for example, a point sensor such as a photodiode or an avalanche photodiode. Alternatively, the light detection element is, for example, an image sensor such as a CCD image sensor or a CMOS image sensor. The preamplifier integrates and amplifies a photocurrent output from the light detection element. The photodetector 12 detects weak measurement light L1 with high sensitivity to generate the detection signal and transmits the detection signal to the main body unit 30 via the cable 18.

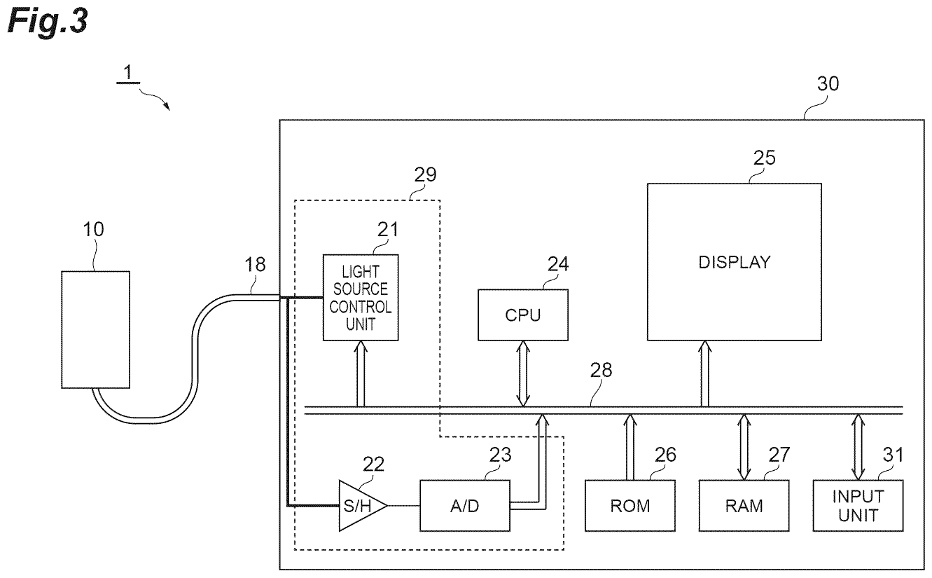

FIG. 3 is a block diagram illustrating a configuration example of the metabolism measurement apparatus 1. The main body unit 30 is a computer having a CPU 24, a display 25, a ROM 26, a RAM 27, a data bus 28, a controller 29, and an input unit 31. The controller 29 includes a light source control unit 21, a sample-and-hold circuit 22, and an analog/digital (A/D) conversion circuit 23.

The light source control unit 21 controls light output from the light measurement instrument 10. The controller 29 controls, for example, an output time interval of the measurement light L1 and the intensity of the measurement light L1. The light source control unit 21 is electrically connected to the data bus 28, and the light source 11 of the light measurement instrument 10. The light source control unit 21 receives an instruction signal for instructing to drive the light source 11 from the CPU 24 by way of the data bus 28. The instruction signal includes information such as the light intensity and the wavelength (for example, any wavelength among the wavelengths λ₁, λ₂, . . . , and λ_{N}) of the measurement light L1 output from the light source 11. The light source control unit 21 drives the light source 11 based on the instruction signal received from the CPU 24. The light source control unit 21 outputs a drive signal to the light measurement instrument 10 via the cable 18.

The sample-and-hold circuit 22 receives and holds the detection signal transmitted from the light measurement instrument 10 via the cable 18. The A/D conversion circuit 23 converts the detection signal into a digital signal and outputs the digital signal to the CPU 24. The sample-and-hold circuit 22 receives a sample signal indicating a timing of holding the detection signal from the CPU 24 via the data bus 28. When the sample signal is received, the sample-and-hold circuit 22 holds the detection signal input from the light measurement instrument 10. The sample-and-hold circuit 22 is electrically connected to the A/D conversion circuit 23 and outputs the held detection signal to the A/D conversion circuit 23.

The CPU 24 is a calculator in the present embodiment. The CPU 24 calculates data regarding the degree of metabolism and/or a blood glucose level of the living body 50 based on the detection signal received from the A/D conversion circuit 23. The CPU 24 transmits the calculated data to the display 25 via the data bus 28. A calculation method of the degree of metabolism based on the detection signal will be described below. The display 25 is electrically connected to the data bus 28 and displays a result transmitted from the CPU 24 via the data bus 28. The display 25 and the input unit 31 may be configured with, for example, a touch panel display.

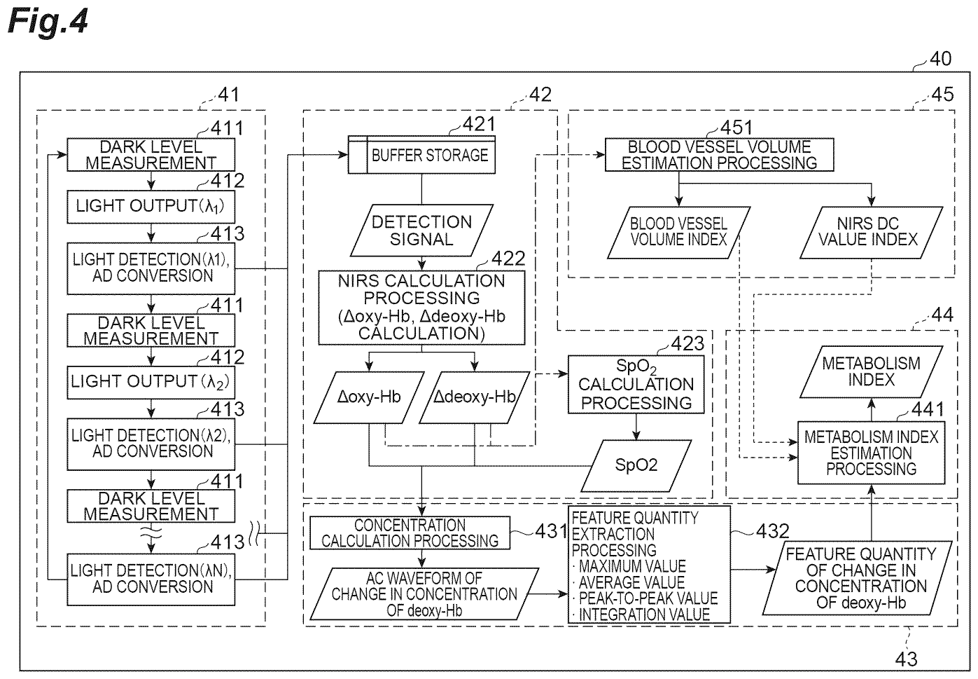

FIG. 4 is a block diagram illustrating functions of a calculator 40 that are implemented by the CPU 24. As illustrated in FIG. 4, the calculator 40 has a measurement control portion 41, a NIRS calculation portion 42 (first calculation portion), a concentration calculation portion 43 (second calculation portion), a metabolism index calculation portion 44 (third calculation portion), and a blood vessel volume calculation portion 45 (fourth calculation portion).

The measurement control portion 41 is implemented by the CPU 24 controlling the controller 29. The measurement control portion 41 repeatedly executes dark level measurement processing 411, light output processing 412, and light detection processing 413 while varying the wavelength of the measurement light L1. In the dark level measurement processing 411, the detection signal from the photodetector 12 is acquired in a state in which the measurement light L1 is not output from the light source 11. With this, a dark current level of the light detection element of the photodetector 12 can be measured. In the light output processing 412, the measurement light L1 is output from the light source 11, and the measurement light L1 is incident on the living body 50. In the light detection processing 413, the measurement light L1 propagating through the living body 50 is detected in the photodetector 12, and the obtained detection signal is converted into the digital signal in the A/D conversion circuit 23. In initial light output processing 412 and light detection processing 413, the measurement light L1 having the wavelength λ, is used. In next light output processing 412 and light detection processing 413, the measurement light L1 having the wavelength λ₂ is used. Hereinafter, the processing is repeated while varying the wavelength of the measurement light L1 until N-th light output processing 412 and light detection processing 413. Although the dark level measurement processing 411 is executed each time the wavelength is varied in the example illustrated in FIG. 4, the dark level measurement processing 411 may be executed only before the initial light output processing 412. The measurement control portion 41 returns to the dark level measurement processing 411 of the wavelength λ₁ after the light detection processing 413 of the wavelength λ_{N}. Then, this operation is repeated until the detection signal reaches a predetermined data length.

The NIRS calculation portion 42 stores the detection signal for each wavelength from the light measurement instrument 10 obtained in the measurement control portion 41, in a buffer 421. The NIRS calculation portion 42 executes NIRS calculation processing 422 based on the detection signal from the light measurement instrument 10. The NIRS calculation processing 422 is processing of obtaining Δoxy-Hb (first parameter) and Δdeoxy-Hb (second parameter) in consideration of an influence such as absorption or scattering of the measurement light L1 due to oxygenated hemoglobin (hereinafter, referred to as O₂Hb) and deoxygenated hemoglobin (hereinafter, referred to as HHb). Δoxy-Hb is a temporal relative change amount from a certain timing (typically, a time when measurement starts) calculated based on a degree of absorption of O₂Hb. Δoxy-Hb is a numerical value depending on a concentration of O₂Hb in blood and a volume of a blood vessel in an optical path. In other words, Δoxy-Hb is a numerical value based on the number of O₂Hb in the optical path. Δdeoxy-Hb is a temporal relative change amount from a certain timing (typically, a time when measurement starts) calculated based on a degree of absorption by HHb. Δdeoxy-Hb is a numerical value depending on a concentration of HHb in blood and the volume of the blood vessel in the optical path. In other words, Δdeoxy-Hb is a numerical value based on the number of HHb in the optical path. In the present specification, the volume of the blood vessel means a total internal volume of the blood vessel in the optical path.

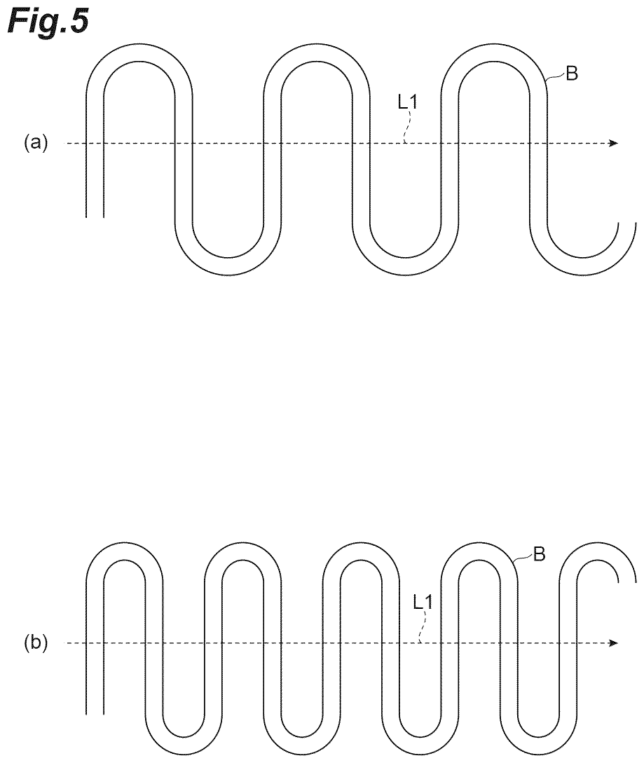

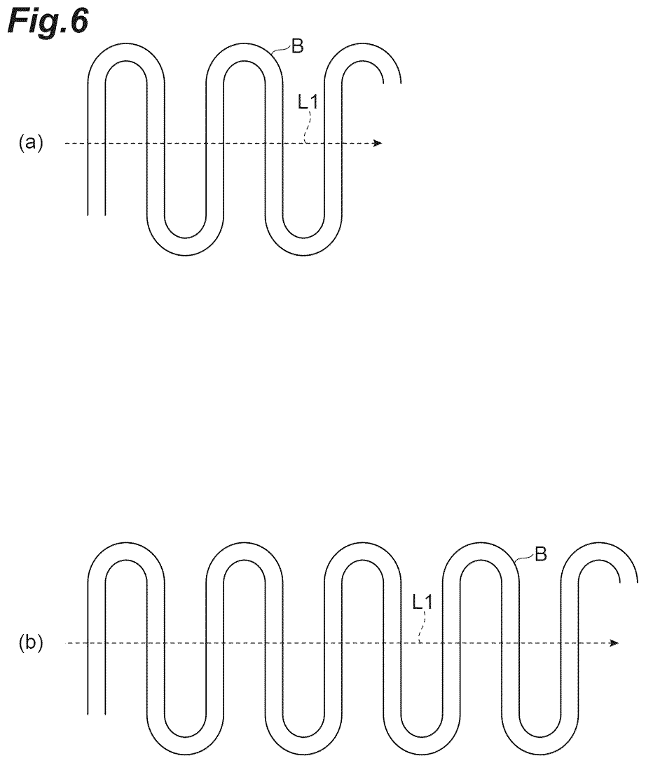

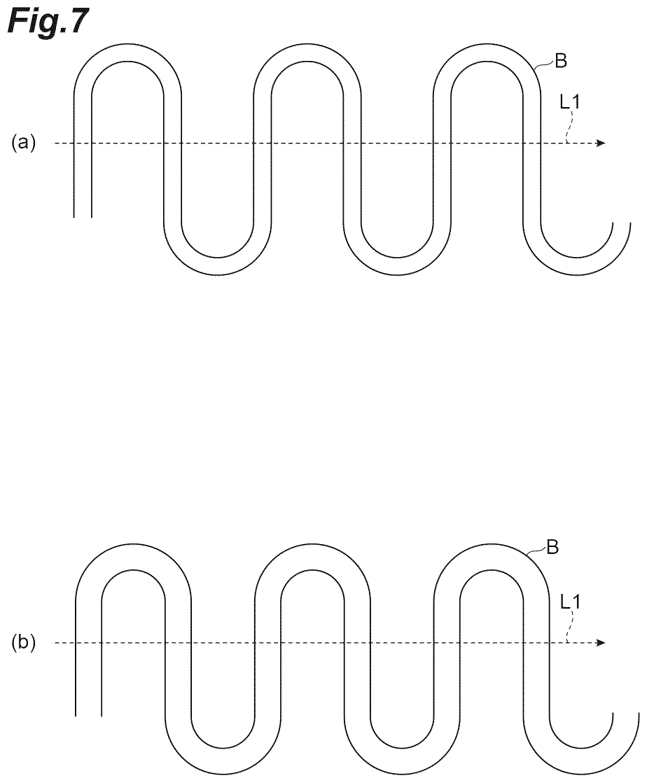

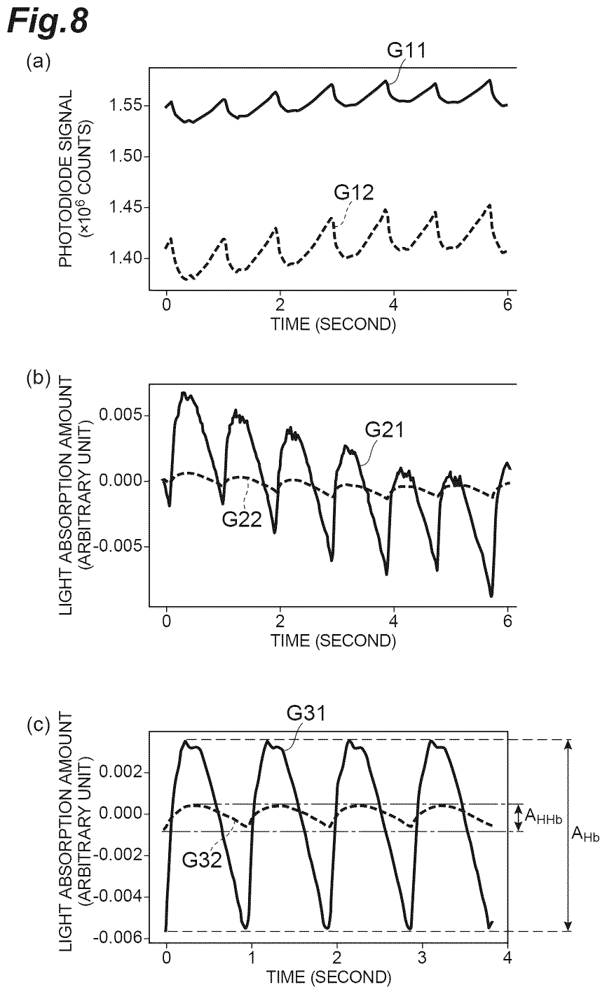

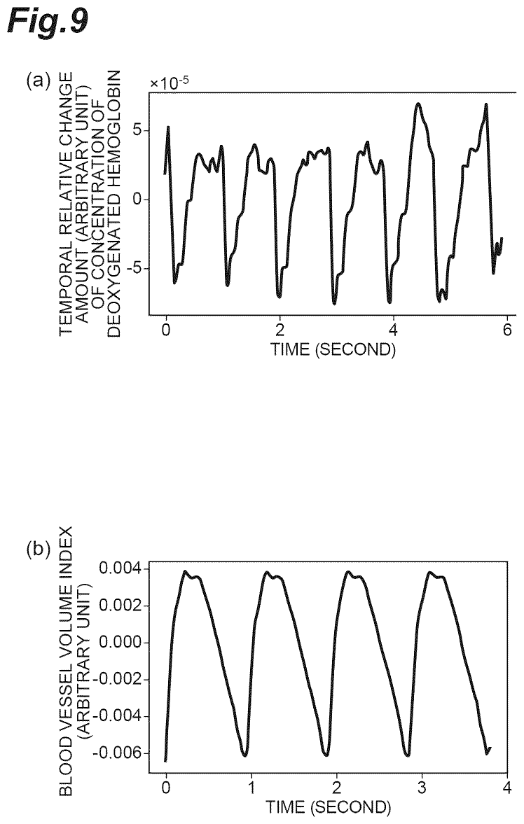

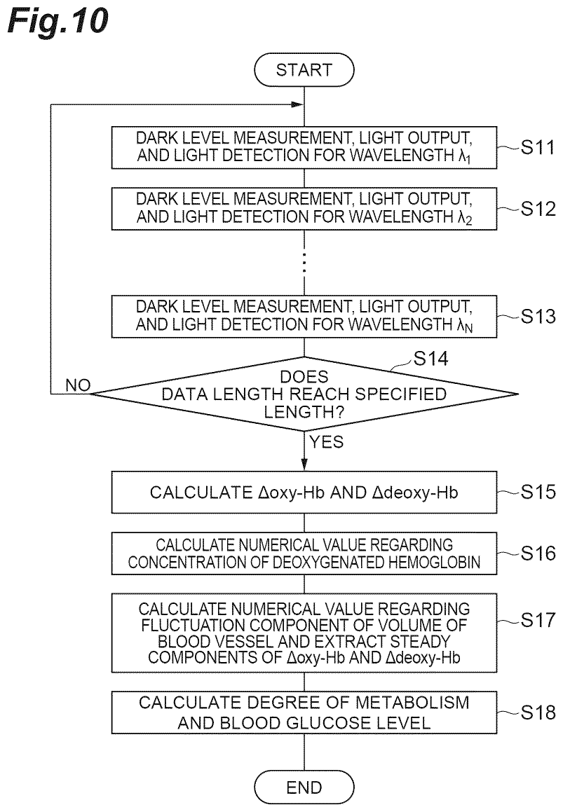

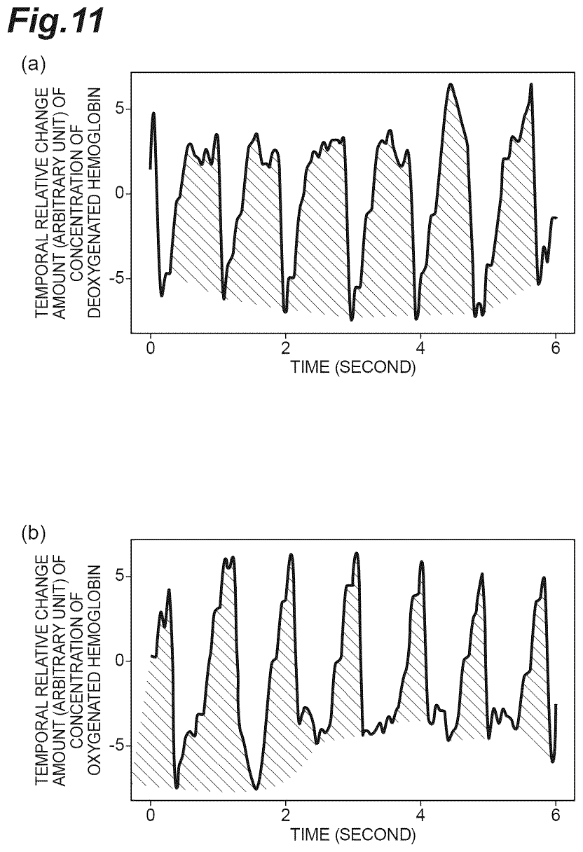

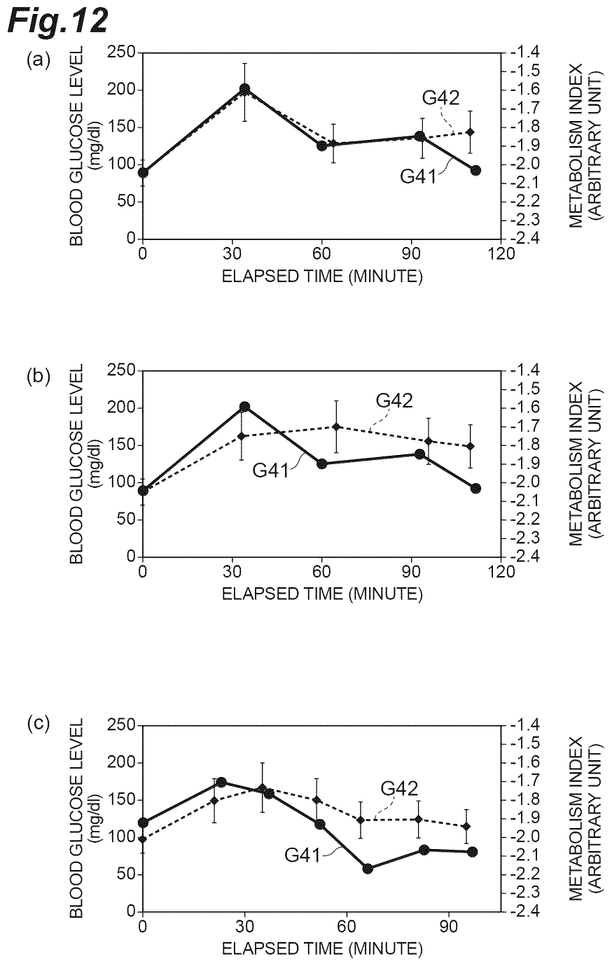

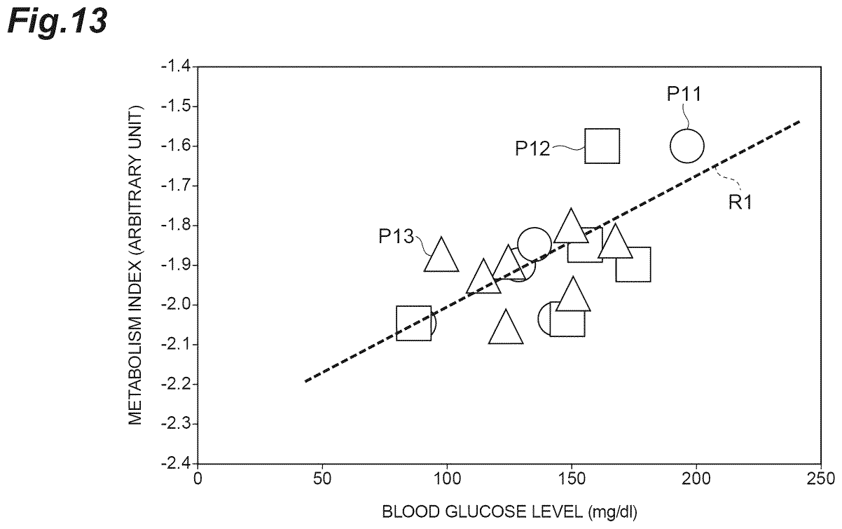

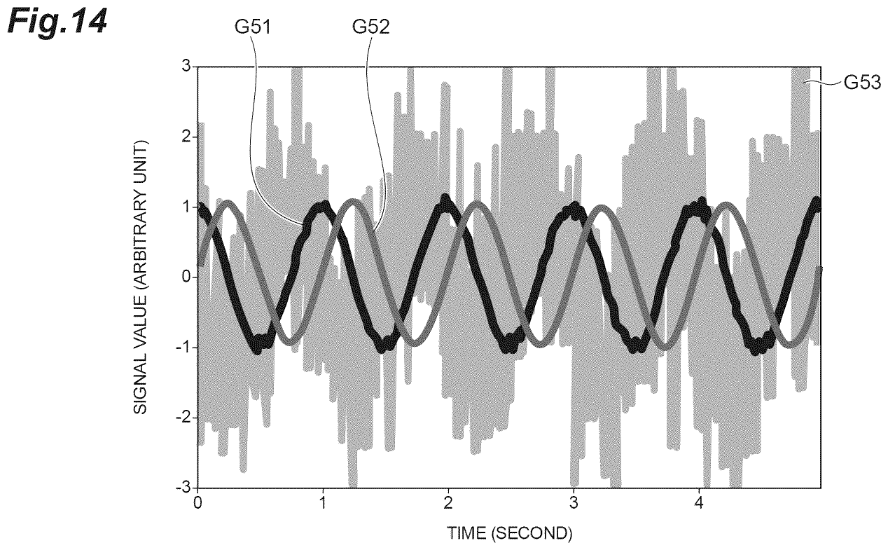

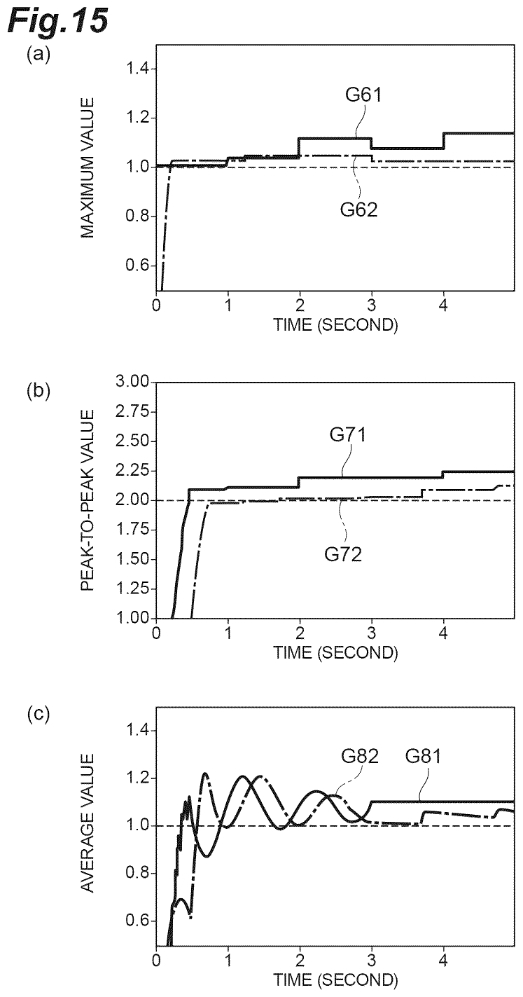

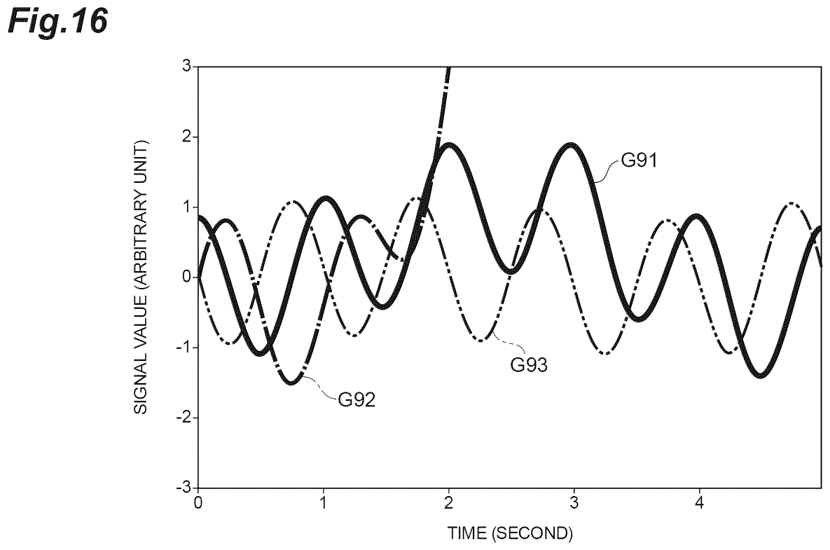

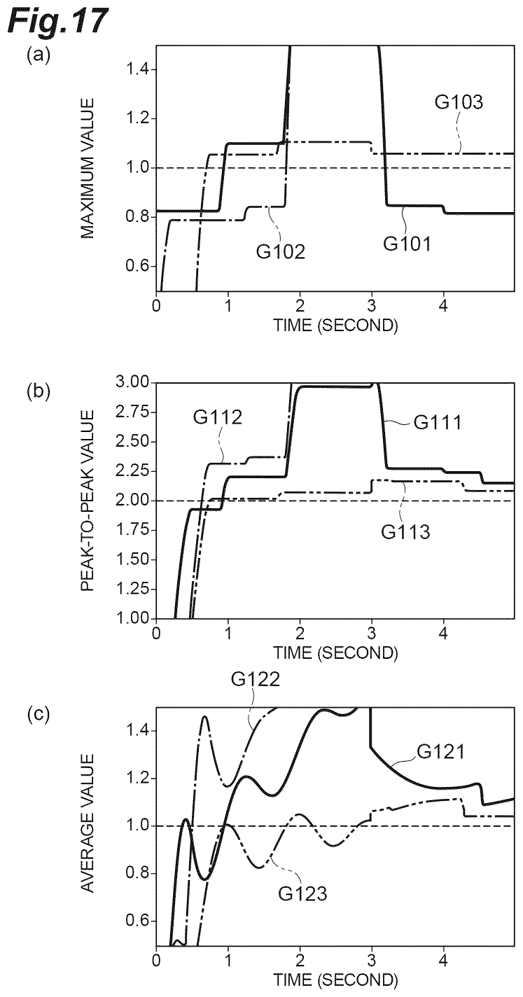

The volume of the blood vessel in the optical path has a close correlation with a blood vessel density, an optical path length, and the thickness of the blood vessel. FIG. 5 and FIG. 6 are schematic views illustrating a volume of a blood vessel in an optical path. Part (a) of FIG. 5 illustrates a case where a density of a blood vessel B is small in a certain optical path of the measurement light L1. Part (b) of FIG. 5 illustrates a case where the density of the blood vessel B is greater than in Part (a) of FIG. 5 in an optical path having the same length as in Part (a) of FIG. 5. When the density of the blood vessel B is small as illustrated in Part (a) of FIG. 5, the number of times the optical path passes through the blood vessel B is small, so that light absorption by hemoglobin is small. On the other hand, when the density of blood vessel B is large as illustrated in Part (b) of FIG. 5, the number of times the optical path passes through the blood vessel B is large, so that light absorption by hemoglobin is large. Part (a) of FIG. 6 illustrates a case where the optical path length of the measurement light L1 is short. Part (b) of FIG. 6 illustrates a case where the density of the blood vessel B is the same as in Part (a) of FIG. 6 and the optical path length of the measurement light L1 is longer than in Part (a) of FIG. 6. When the optical path length of the measurement light L1 is short as illustrated in Part (a) of FIG. 6, the number of times the optical path passes through the blood vessel B is small, so that light absorption by hemoglobin is small. On the other hand, when the optical path length of the measurement light L1 is long as illustrated in Part (b) of FIG. 6, the number of times the optical path passes through the blood vessel B is large, so that light absorption by hemoglobin is large. Part (a) of FIG. 7 illustrates a case where the blood vessel B is thin. Part (b) of FIG. 7 illustrates a case where the blood vessel B is thicker than in Part (a) of FIG. 7. When the blood vessel B is thin as illustrated in Part (a) of FIG. 7, a distance at which the optical path passes through the blood vessel B is short, so that light absorption by hemoglobin is small. On the other hand, when the blood vessel B is thick as illustrated in Part (b) of FIG. 7, the distance at which the optical path passes through the blood vessel B is long, so that light absorption by hemoglobin is large.

That is, as the density of the blood vessel in the optical path is greater, the volume of the blood vessel is greater, and as the density of the blood vessel in the optical path is smaller, the volume of the blood vessel is smaller. As the optical path length of the measurement light L1 is longer, the volume of the blood vessel is greater, and as the optical path length of the measurement light L1 is shorter, the volume of the blood vessel is smaller. As an average diameter of the blood vessel in the optical path is greater, the volume of the blood vessel is greater, and as the average diameter of the blood vessel in the optical path is smaller, the volume of the blood vessel is smaller.

A calculation method of Δoxy-Hb and Δdeoxy-Hb will be described in detail. In the description, the number N of wavelengths (λ₁, λ₂, . . . , and λ_{N}) of the measurement light L1 is set to three. At a certain light detection position, values of the detection signals according to the respective measurement light wavelengths λ₁ to λ₃ at a time T₀ are referred to as Dλ₁(T₀) to Dλ₃(T₀), and the values at a time T₁ are referred to as Dλ₁(T₁) to Dλ₃(T₁). A change amount of detected light intensity at the times T₀ to T₁ is represented as Formulas (3) to (5).

$$\begin{matrix}[ Formula 3 ] & \\ Δ {OD}_{1} ( {T}_{1} ) = log ( \frac{{D}_{λ 1} ( {T}_{1} )}{{D}_{λ 1} ( {T}_{0} )} ) & ( 3 )\end{matrix}$$

$$\begin{matrix}[ Formula 4 ] & \\ Δ {OD}_{2} ( {T}_{1} ) = log ( \frac{{D}_{λ 2} ( {T}_{1} )}{{D}_{λ 2} ( {T}_{0} )} ) & ( 4 )\end{matrix}$$

$$\begin{matrix}[ Formula 5 ] & \\ Δ {OD}_{3} ( {T}_{1} ) = log ( \frac{{D}_{λ3} ( {T}_{1} )}{{D}_{λ3} ( {T}_{0} )} ) & ( 5 )\end{matrix}$$

In Formulas (3) to (5), ΔOD₁(T₁) is a temporal relative change amount from the time T₀ of the detected light intensity at the wavelength λ₁, ΔOD₂(T₁) is a temporal relative change amount from the time T₀ of the detected light intensity at the wavelength λ₂, and ΔOD₃(T₁) is a temporal relative change amount from the time T₀ of the detected light intensity at the wavelength λ₃. Part (a) of FIG. 8 is a graph illustrating an example of change (graph G11) over time in detected light intensity at the wavelength of 660 mm and an example of change (graph G12) over time in detected light intensity at the wavelength of 910 mm. In Part (a) of FIG. 8, the horizontal axis indicates time (second), and the vertical axis indicates a detection signal (×10⁶ counts).

Δoxy-Hb and Δdeoxy-Hb from the time T₀ to the time T₁ are referred to as N_{oxy}(T₁) and N_{deoxy}(T₁), respectively. These can be obtained by Formula (6).

$$\begin{matrix}[ Formula 6 ] & \\ ( \begin{matrix}{N}_{oxy} ( {T}_{1} ) \\ {N}_{deoxy} ( {T}_{1} )\end{matrix} ) = ( \begin{matrix}{a}_{11} & {a}_{12} & {a}_{13} \\ {a}_{21} & {a}_{22} & {a}_{23}\end{matrix} ) ( \begin{matrix}Δ {OD}_{1} ( {T}_{1} ) \\ Δ {OD}_{2} ( {T}_{1} ) \\ Δ {OD}_{3} ( {T}_{1} )\end{matrix} ) & ( 6 )\end{matrix}$$

In Formula (6), coefficients a₁₁ to a₂₃ are constants that are obtained from absorption coefficients of O₂Hb and HHb with respect to light having the wavelengths λ₁, λ₂, and λ₃. The NIRS calculation portion 42 periodically performs such calculation even after the time T₁. The NIRS calculation portion 42 periodically calculates Δoxy-Hb and Δdeoxy-Hb at each time T₁, T₂, T₃, . . . by such calculation. A calculation period of Δoxy-Hb and Δdeoxy-Hb is, for example, 16 milliseconds. Part (b) of FIG. 8 is a graph illustrating an example of change (graph G21) over time in Δoxy-Hb at the wavelength of 660 mm and an example of change (graph G22) over time in Δdeoxy-Hb at the wavelength of 910 mm. In Part (b) of FIG. 8, the horizontal axis indicates time (second), and the vertical axis indicates a light absorption amount (arbitrary unit).

The NIRS calculation portion 42 executes SpO₂ calculation processing 423 of calculating an oxygen saturation (SpO₂) from Δoxy-Hb and Δdeoxy-Hb. Part (c) of FIG. 8 is a graph illustrating a result of executing filter processing of removing a low-frequency component smaller than a heartbeat frequency on the graph illustrated in Part (b) of FIG. 8. In Part (c) of FIG. 8, the horizontal axis indicates time (second), and the vertical axis indicates a light absorption amount (arbitrary unit). SpO₂ is obtained by, for example, Formula (7) described below based on amplitude A_{Hb} of Δoxy-Hb and amplitude Δ_{HHb} of Δdeoxy-Hb illustrated in Part (c) of FIG. 8. In general, SpO₂ means a value of an oxygen saturation in arterial blood estimated from pulse wave amplitude. However, in the present specification, SpO₂ means a value of a local oxygen saturation at a measurement site including an artery, a peripheral blood vessel, and the like.

$$\begin{matrix}[ Formula 7 ] & \\ {SpO}_{2} = \frac{{A}_{Hb}}{{A}_{Hb} + {A}_{HHb}} & ( 7 )\end{matrix}$$

The concentration calculation portion 43 executes concentration calculation processing 431 based on Δoxy-Hb and Δdeoxy-Hb calculated in the NIRS calculation portion 42. The concentration calculation processing 431 is processing of obtaining a numerical value (fourth parameter) regarding a concentration of HHb in blood from which an influence of the volume of the blood vessel in the optical path is excluded. The numerical value regarding the concentration of HHb in blood is, for example, a temporal relative change amount of the concentration of HHb from a certain timing, typically, the time T₀ that is a time when measurement starts.

Part (a) of FIG. 9 is a graph illustrating an example of change over time in the temporal relative change amount of the concentration of HHb. In Part (a) of FIG. 9, the horizontal axis indicates time (second), and the vertical axis indicates the temporal relative change amount (arbitrary unit) of the concentration of HHb.

A method for obtaining the temporal relative change amount of the concentration of HHb from a certain timing will be described in detail. Here, the following (1) to (3) are set as preconditions.

(1) A time waveform of each of Δoxy-Hb and Δdeoxy-Hb calculated by the NIRS calculation portion 42 is proportional to a product of the volume of the blood vessel and each concentration of hemoglobin.

(2) The time waveform of each of Δoxy-Hb and Δdeoxy-Hb has a steady component (direct-current component) that is not changed temporally and a fluctuation component (alternating-current component) that fluctuates in a period according to the heartbeat frequency and harmonics thereof. There is no undulation component of a low frequency within a range of 0 Hz to the heartbeat frequency.

(3) Within a short time from a measurement start time, an amount of moisture in the blood vessel is substantially constant, and a total concentration of hemoglobin is constant.

Formula (8) described below is given from the above-described precondition (1). C_{oxy}(t) is a concentration of O₂Hb at a time t, C_{deoxy}(t) is a concentration of HHb at the time t, and V(t) is the volume of the blood vessel in the optical path.

$$\begin{matrix}[ Formula 8 ] & \\ \{ \begin{matrix}{N}_{oxy} ( t ) ∝ {C}_{oxy} ( t ) \cdot V ( t ) \\ {N}_{deoxy} ( t ) ∝ {C}_{deoxy} ( t ) \cdot V ( t )\end{matrix} & ( 8 )\end{matrix}$$

Formulas (9) to (11) described below are given from the above-described precondition (2). C_{oxy,DC} and C_{deoxy,DC} are steady components of the concentration of O₂Hb and the concentration of HHb, respectively. C_{oxy,AC}(t) and C_{deoxy,AC}(t) are fluctuation components of the concentration of O₂Hb and the concentration of HHb, respectively. V_{DC} is a steady component of the volume of the blood vessel. V_{AC}(t) is a fluctuation component of the volume of the blood vessel. t₀ is the measurement start time, and T is a time interval from the measurement start time to a current time.

$$\begin{matrix}[ Formula 9 ] & \\ \{ \begin{matrix}{C}_{oxy} ( t ) = {C}_{oxy , D C} + {C}_{oxy , A C} ( t ) \\ {C}_{deoxy} ( t ) = {C}_{deoxy , D C} + {C}_{deoxy , A C} ( t )\end{matrix} & ( 9 )\end{matrix}$$

$$\begin{matrix}[ Formula 10 ] & \\ \{ \begin{matrix}{\int }_{{t}_{0}}^{{t}_{0} + T} {C}_{oxy , A C} ( t ) dt = 0 \\ {\int }_{{t}_{0}}^{{t}_{0} + T} {C}_{deoxy , A C} ( t ) dt = 0\end{matrix} & ( 10 )\end{matrix}$$

$$\begin{matrix}[ Formula 11 ] & \\ V ( t ) = {V}_{D C} + {V}_{A C} ( t ) & ( 11 )\end{matrix}$$

Formula (12) described below is given from the above-described precondition (3). const. means a constant.

$$\begin{matrix}[ Formula 12 ] & \\ \{ \begin{matrix}{C}_{oxy} ( t ) + {C}_{deoxy} ( t ) = const . \\ {C}_{oxy , A C} ( t ) + {C}_{deoxy , A C} ( t ) = 0\end{matrix} & ( 12 )\end{matrix}$$

Formula (13) is obtained from Formulas (8), (9), and (11).

$$\begin{matrix}[ Formula 13 ] & \\ {N}_{oxy} ( t ) ∝ {C}_{oxy} ( t ) \cdot V ( t ) = {C}_{oxy , D C} \cdot {V}_{D C} + {C}_{oxy , D C} \cdot {V}_{A C} ( t ) + {C}_{oxy , A C} ( t ) \cdot {V}_{D C} + {C}_{oxy , A C} ( t ) \cdot {V}_{A C} ( t ) & ( 13 )\end{matrix}$$

When Formula (12) is applied to Formula (13), Formula (14) described below is obtained.

$$\begin{matrix}[ Formula 14 ] & \\ {N}_{oxy} ( t ) + {N}_{deoxy} ( t ) ∝ [ {C}_{oxy} ( t ) + {C}_{deoxy} ( t ) ] \cdot V ( t ) = ( {C}_{oxy , D C} + {C}_{deoxy , D C} ) \cdot [ {V}_{D C} + {V}_{A C} ( t ) ] & ( 14 )\end{matrix}$$

In Formula (14), when time differentiation is performed on both sides to remove a constant term, and a proper proportionality coefficient α is set, Formula (15) described below is obtained. In Formula (15), a dot attached above a character represents time differentiation.

$$\begin{matrix}[ Formula 15 ] & \\ α [ {\dot{N}}_{oxy} ( t ) + {\dot{N}}_{deoxy} ( t ) ] = ( {C}_{oxy , D C} + {C}_{deoxy , D C} ) \cdot {\dot{V}}_{A C} ( t ) & ( 15 )\end{matrix}$$

In this way, a sum of the time waveforms of Δoxy-Hb and Δdeoxy-Hb obtained by the NIRS calculation portion 42 is taken and an AC component thereof is extracted, so that the influence of the concentration of hemoglobin can be excluded and the fluctuation component V_{AC}(t) of the volume of the blood vessel can be taken out.

Similarly to Formula (13) regarding N_{oxy}(t), Formula (16) regarding N_{deoxy}(t) is established.

$$\begin{matrix}[ Formula 16 ] & \\ {N}_{deoxy} ( t ) ∝ {C}_{deoxy , D C} \cdot {V}_{D C} + {C}_{deoxy , D C} \cdot {V}_{A C} ( t ) + {C}_{deoxy , A C} ( t ) \cdot {V}_{D C} + {C}_{deoxy , A C} ( t ) \cdot {V}_{A C} ( t ) & ( 16 )\end{matrix}$$

The proportionality coefficient α used in Formula (15) is common to both N_{oxy}(t) and N_{deoxy}(t). Accordingly, when Formula (16) is rearranged, Formula (17) described below is obtained.

$$\begin{matrix}[ Formula 17 ] & \\ α \cdot {N}_{deoxy} ( t ) ∝ [ {C}_{deoxy , D C} + {C}_{deoxy , A C} ( t ) ] \cdot {V}_{A C} ( t ) + [ {C}_{deoxy , D C} + {C}_{deoxy , A C} ( t ) ] \cdot {V}_{D C} & ( 17 )\end{matrix}$$

In Formula (17) described above, when time differentiation is performed on both sides to remove a constant term, Formula (18) described below is obtained.

$$\begin{matrix}[ Formula 18 ] & \\ α \cdot {\dot{N}}_{deoxy} ( t ) = [ {C}_{deoxy , D C} + {C}_{deoxy , A C} ( t ) ] \cdot {\dot{V}}_{A C} ( t ) + {\dot{C}}_{deoxy , A C} ( t ) \cdot [ {V}_{D C} + {V}_{A C} ( t ) ] & ( 18 )\end{matrix}$$

When Formula (15) is applied to remove a differentiation term of V_{AC}(t) in Formula (18), Formula (19) described below is obtained.

$$[ Formula 19 ]$$

$$α \cdot {\dot{N}}_{deoxy} ( t ) = [ {C}_{deoxy , DC} + {C}_{deoxy , AC} ( t ) ] \cdot \frac{α [ {\dot{N}}_{oxy} ( t ) + {\dot{N}}_{deoxy} ( t ) ]}{{C}_{oxy , DC} + {C}_{deoxy , DC}} + {\dot{C}}_{deoxy , AC} ( t ) \cdot [ {V}_{DC} + {V}_{AC} ( t ) ] \qquad ( 19 )$$

In Formula (19), when a differentiation term of C_{deoxy,AC}(t) is arranged, Formula (20) described below is obtained.

$$[ Formula 20 ]$$

$${\dot{C}}_{deoxy , AC} ( t ) = \frac{α}{{V}_{DC} + {V}_{AC} ( t )} \cdot \{ {\dot{N}}_{deoxy} ( t ) - \frac{{C}_{deoxy , DC} + {C}_{deoxy , AC} ( t )}{{C}_{oxy , DC} + {C}_{deoxy , DC}} \cdot [ {\dot{N}}_{oxy} ( t ) + {\dot{N}}_{deoxy} ( t ) ] \} \qquad ( 20 )$$

In Formula (20), an expression is simplified on a further assumption. First, it is considered that fluctuation of the volume of the blood vessel due to the beat is small compared to an average value of the volume of the blood vessel. Accordingly, approximation of Formula (21) described below is performed.

$$[ Formula 21 ]$$

$${V}_{DC} + {V}_{AC} ( t ) \text{?} {V}_{DC} = const . \qquad ( 21 )$$

$$\text{?} \text{indicates text missing or illegible when filed}$$

It is also considered that fluctuation of the concentration of deoxyhemoglobin due to the beat is small compared to an average value of the concentration of deoxyhemoglobin. Accordingly, approximation illustrated in Formula (22) described below is performed.

$$[ Formula 22 ]$$

$${C}_{deoxy , DC} + {C}_{deoxy , AC} ( t ) \approx {C}_{deoxy , DC} \text{?} const . \qquad ( 22 )$$

$$\text{?} \text{indicates text missing or illegible when filed}$$

When the approximation of Formulas (21) and (22) are applied to Formula (20), Formula (23) described below is obtained.

$$[ Formula 23 ]$$

$${\dot{C}}_{deoxy , AC} ( t ) \approx \frac{α}{{V}_{DC}} \cdot \{ \frac{{C}_{oxy , DC}}{{C}_{oxy , DC} + {C}_{deoxy , DC}} \cdot {\dot{N}}_{deoxy} ( t ) - \frac{{C}_{deoxy , DC}}{{C}_{oxy , DC} + {C}_{deoxy , DC}} \cdot {\dot{N}}_{oxy} ( t ) \} \qquad ( 23 )$$

Here, the oxygen saturation SpO₂ is represented by Formula (24) described below.

$$[ Formula 24 ]$$

$${Sp O}_{2} = \frac{{C}_{oxy , DC}}{{C}_{oxy , DC} + {C}_{deoxy , DC}} \qquad ( 24 )$$

When Formula (24) is applied to Formula (23), Formula (25) is obtained.

$$[ Formula 25 ]$$

$${\dot{C}}_{deoxy , AC} ( t ) \approx \frac{α}{{V}_{DC}} \cdot \{ {Sp O}_{2} \cdot {\dot{N}}_{deoxy} ( t ) - ( 1 - {Sp O}_{2} ) \cdot {\dot{N}}_{oxy} ( t ) \} \qquad ( 25 )$$

When it is assumed that a primitive function C_{deoxy,AC}(t) of a left side of Formula (25) is a periodic function in which an average value is zero, a constant term is 0, so that Formula (26) obtained by performing time integration on both sides of Formula (25) is also established similarly.

$$[ Formula 26 ]$$

$${C}_{deoxy , AC} ( t ) \approx \frac{α}{{V}_{DC}} \cdot \{ {Sp O}_{2} \cdot {N}_{deoxy} ( t ) - ( 1 - {Sp O}_{2} ) \cdot {N}_{oxy} ( t ) \} \qquad ( 26 )$$

When Formula (26) is rearranged, Formula (27) regarding C_{deoxy,AC}(t), N_{oxy}(t), N_{deoxy}(t), and SpO₂ is obtained.

$$[ Formula 27 ]$$

$${Sp O}_{2} \cdot {N}_{deoxy} ( t ) - ( 1 - {Sp O}_{2} ) \cdot {N}_{oxy} ( t ) \approx \frac{{V}_{DC}}{α} \cdot {C}_{deoxy , AC} ( t ) \qquad ( 27 )$$

When there is no attachment or detachment of the light measurement instrument 10, fluctuation of the blood flow, or the like, the volume V(t) of the blood vessel in the optical path can be assumed to be constant within a certain determined period (for example, several minutes to several months). Accordingly, (α/V_{DC}) can be considered as a constant. In an example, the volume V(t) of the blood vessel is assumed to be constant. In this case, the blood vessel volume calculation portion 45 described below is not required. There is a case where a drift of the steady component V_{DC} due to the fluctuation of the blood flow and/or an offset of the steady component V_{DC} caused by the attachment or detachment of the light measurement instrument 10 can be suppressed in terms of engineering. For example, there is a case where a shape of the light measurement instrument 10 is optimized such that an attachment position and an attachment angle of the light measurement instrument 10 are the same every time. In such a case, even though there is the attachment or detachment of the light measurement instrument 10, the fluctuation of the blood flow, or the like, there is a case where (α/V_{DC}) can be considered to be constant.

As described above, each of N_{oxy}(t) and N_{deoxy}(t) obtained in the NIRS calculation portion 42 is a numerical value depending on each concentration of hemoglobin in blood and the volume of the blood vessel in the optical path. However, as described above, predetermined calculation processing is executed on N_{oxy}(t) and N_{deoxy}(t), so that a numerical value regarding only the concentration of HHb in blood from which the influence of the volume of the blood vessel in the optical path is excluded can be obtained. The concentration calculation portion 43 can calculate, for example, the temporal relative change amount of the concentration of HHb based on Formula (27) described above as the numerical value regarding the concentration of HHb.

For example, when actual C_{oxy}(t) and C_{deoxy}(t) or a total concentration C_{oxy}(t)+C_{deoxy}(t) of hemoglobin that is a sum of C_{oxy}(t) and C_{deoxy}(t) is measured by a method such as blood collection at the measurement start time, a value of the constant (α/V_{DC}) can be obtained by comparison with the numerical value obtained by Formula (27). In this case, the concentration calculation portion 43 can successively calculate an absolute amount of the concentration of HHb based on Formula (27) described above.

The concentration calculation portion 43 of the present embodiment further executes feature quantity extraction processing 432 of extracting a quantity of predetermined feature from the numerical value regarding the concentration of HHb. The quantity of predetermined feature is, for example, at least one value selected from a group consisting of a maximum value, a time average value, a peak-to-peak value, and a time integration value of the numerical value regarding the concentration of HHb.

The metabolism index calculation portion 44 executes metabolism index estimation processing 441 based on a predetermined relationship between the numerical value regarding the concentration of HHb and the degree of metabolism. The metabolism index estimation processing 441 is processing of obtaining data (metabolism index) regarding the degree of metabolism. In the metabolism index estimation processing 441 of the present embodiment, data (metabolism index) regarding the degree of metabolism is obtained based on a pre-acquired relationship between the quantity of predetermined feature from the concentration calculation portion 43 and the degree of metabolism. A data table regarding the relationship between the quantity of predetermined feature and the degree of metabolism is stored in, for example, the ROM 26 illustrated in FIG. 3.

The metabolism index calculation portion 44 may further obtain a blood glucose level of the living body 50 based on a pre-acquired relationship between the degree of metabolism and the blood glucose level. A data table regarding the relationship between the degree of metabolism and the blood glucose level is stored in, for example, the ROM 26 illustrated in FIG. 3. Because there are individual differences in the relationship between the degree of metabolism and the blood glucose level, the data table may be prepared for each subject. The pre-acquired relationship between the degree of metabolism and the blood glucose level may be appropriately corrected according to a change in a metabolic rate due to age and/or a change in a metabolic function due to the progress of a disease such as diabetes. Specifically, the relationship between the degree of metabolism and the blood glucose level may be corrected according to at least one parameter among the age, sex, height, and the degree of progress of a disease of the subject.

Subsequently, the blood vessel volume calculation portion 45 will be described. The blood vessel volume calculation portion 45 executes blood vessel volume estimation processing 451. The blood vessel volume estimation processing 451 is processing of obtaining a value for correcting a blood vessel volume index, that is, the steady component V_{DC} (see Formula (27)) of the volume of the blood vessel in data regarding the degree of metabolism from Δoxy-Hb and Δdeoxy-Hb obtained in the NIRS calculation portion 42. The metabolism index calculation portion 44 corrects data regarding the degree of metabolism based on the value obtained by the blood vessel volume calculation portion 45. With this, even though there is the attachment or detachment of the light measurement instrument 10, the fluctuation of the blood flow, or the like, data regarding the degree of metabolism can be obtained with high reproducibility.

When Formula (15) described above is rearranged, Formula (28) described below is obtained.

$$[ Formula 28 ]$$

$${\dot{N}}_{oxy} ( t ) + {\dot{N}}_{deoxy} ( t ) \frac{1}{α} ( {C}_{oxy , DC} + {C}_{deoxy , DC} ) \cdot {\dot{V}}_{AC} ( t ) \qquad ( 28 )$$

When time integration is performed on both sides of Formula (28), and thereafter, only a periodic fluctuation component is further taken out, Formula (29) described below is obtained.

$$[ Formula 29 ]$$

$${\tilde{N}}_{oxy} ( t ) + {\tilde{N}}_{deoxy} ( t ) = \frac{1}{α} ( {C}_{oxy , DC} + {C}_{deoxy , DC} ) \cdot {V}_{AC} ( t ) \qquad ( 29 )$$

That is, a sum of Δoxy-Hb and Δdeoxy-Hb obtained in the NIRS calculation portion 42 is proportional to the fluctuation component V_{AC}(t) of the volume of the blood vessel. It is considered that the amplitude of the fluctuation component V_{AC}(t) of the volume of the blood vessel has a strong correlation with the steady component V_{DC} of the volume of the blood vessel. The blood vessel volume calculation portion 45 calculates a numerical value regarding the fluctuation component V_{AC}(t) of the volume of the blood vessel from Formula (29). Then, the blood vessel volume calculation portion 45 obtains the value for correcting the steady component V_{DC} of the volume of the blood vessel in data regarding the degree of metabolism based on a predetermined relationship between the numerical value and the steady component V_{DC} of the volume of the blood vessel. In this case, even though the drift of the steady component V_{DC} due to the fluctuation of the blood flow and/or the offset of the steady component V_{DC} caused by the attachment or detachment of the light measurement instrument 10 is not suppressed in terms of engineering, the steady component V_{DC} can be corrected from Δoxy-Hb and Δdeoxy-Hb by numerical calculation. A data table regarding the predetermined relationship between the numerical value regarding the fluctuation component V_{AC}(t) of the volume of the blood vessel and the steady component V_{DC} of the volume of the blood vessel is stored in, for example, the ROM 26 illustrated in FIG. 3.

The blood vessel volume index may have a value of a right side of Formula (29). Part (b) of FIG. 9 is a graph illustrating an example of change over time in the value of the right side of Formula (29). In Part (b) of FIG. 9, the horizontal axis indicates time (second), and the vertical axis indicates the value of the right side of Formula (29), that is, a value of Formula (30).

$$[ Formula 30 ]$$

$$\frac{1}{α} ( {C}_{oxy , DC} + {C}_{deoxy , DC} ) \cdot {V}_{AC} ( t ) \qquad ( 30 )$$

The value of Formula (30) is equal to the AC component of the sum of Δoxy-Hb and Δdeoxy-Hb.

The blood vessel volume index may be obtained by extracting a quantity of predetermined feature from the value of Formula (30). The quantity of predetermined feature is, for example, at least one value selected from a group consisting of a maximum value, a time average value, a peak-to-peak value, and a time integration value of the value of Formula (30).

The blood vessel volume calculation portion 45 may extract the steady components of Δoxy-Hb and Δdeoxy-Hb, that is, steady components of N_{oxy}(t) and N_{deoxy}(t) by, for example, low-pass filter processing in the blood vessel volume estimation processing 451. It is considered that the steady components of Δoxy-Hb and Δdeoxy-Hb have a strong correlation with the steady component V_{DC} of the volume of the blood vessel. For this reason, the blood vessel volume calculation portion 45 can obtain an NIRS DC value index, that is, the value for correcting the steady component V_{DC} of the volume of the blood vessel in data regarding the degree of metabolism based on a predetermined relationship between the steady components of Δoxy-Hb and Δdeoxy-Hb and the steady component V_{DC} of the volume of the blood vessel. Accordingly, also in this case, even though the drift of the steady component V_{DC} due to the fluctuation of the blood flow and/or the offset of the steady component V_{DC} caused by the attachment or detachment of the light measurement instrument 10 is not suppressed in terms of engineering, the steady component V_{DC} can be corrected from Δoxy-Hb and Δdeoxy-Hb by numerical calculation.

Next, the operation of the metabolism measurement apparatus 1 will be described. Besides, the metabolism calculation method according to the present embodiment will be described. The metabolism calculation method is suitably executed by the CPU 24 reading and executing a program stored in a non-transitory storage medium, such as the ROM 26, for example. FIG. 10 is a flowchart illustrating the metabolism calculation method according to the present embodiment. In the following description, FIG. 4 is also referred to.

First, in Step S11, the wavelength of the measurement light L1 is set to λ₁, and the dark level measurement processing 411, the light output processing 412, and the light detection processing 413 are executed. Details of the dark level measurement processing 411, the light output processing 412, and the light detection processing 413 are as described above. Next, in Step S12, the wavelength of the measurement light L1 is set to λ₂, and the dark level measurement processing 411, the light output processing 412, and the light detection processing 413 are executed again. In this way, the dark level measurement processing 411, the light output processing 412, and the light detection processing 413 are repeatedly executed while varying the wavelength to the wavelength λ_{N} of the measurement light L1 (Step S13). Then, Steps S11 to S13 are repeated until the data length of the detection signal stored in the buffer 421 reaches a specified length (in other words, a predetermined data length) (Step S14: NO).

After the data length of the detection signal stored in the buffer 421 reaches the specified length (Step S14: YES), Δoxy-Hb and Δdeoxy-Hb are calculated by the NIRS calculation processing 422 (Step S15, first calculation step). In addition, in Step S15, the oxygen saturation (SpO₂) is calculated from Δoxy-Hb and Δdeoxy-Hb by the SpO₂ calculation processing 423. Details of the NIRS calculation processing 422 and the SpO₂ calculation processing 423 are as described above.

After Step S15, the numerical value regarding the concentration of HHb in blood from which the influence of the volume of the blood vessel in the optical path is excluded is obtained by the concentration calculation processing 431 (Step S16, second calculation step). The numerical value regarding the concentration of HHb in blood is, for example, the temporal relative change amount of the concentration of HHb from a certain timing, typically, the time when measurement starts. In addition, in Step S16, the quantity of predetermined feature is extracted from the numerical value regarding the concentration of HHb by the feature quantity extraction processing 432. Details of the concentration calculation processing 431 and the feature quantity extraction processing 432 are as described above.

After Step S16, the value (blood vessel volume index) for correcting the steady component V_{DC} of the volume of the blood vessel in data regarding the degree of metabolism is obtained from Δoxy-Hb and Δdeoxy-Hb obtained in the NIRS calculation portion 42 by the blood vessel volume estimation processing 451 (Step S17, fourth calculation step). In addition, in Step S17, the steady components of Δoxy-Hb and Δdeoxy-Hb, that is, the steady components of N_{oxy}(t) and N_{deoxy}(t) are extracted by, for example, low-pass filter processing by the blood vessel volume estimation processing 451. Any one of the processing of obtaining the blood vessel volume index and the processing of extracting the steady components of Δoxy-Hb and Δdeoxy-Hb may be executed or both processing may be executed. Details of the blood vessel volume estimation processing 451 are as described above.

After Step S17, data (metabolism index) regarding the degree of metabolism is obtained based on the predetermined relationship between the numerical value regarding the concentration of HHb and the degree of metabolism by the metabolism index estimation processing 441 (Step S18, third calculation step). In Step S17, the blood glucose level of the living body 50 may be further obtained based on the pre-acquired relationship between the degree of metabolism and the blood glucose level. Details of the metabolism index estimation processing 441 and the calculation of the blood glucose level are as described above.

In the above description, the concentration calculation portion 43 calculates the numerical value regarding the concentration of HHb, and the metabolism index calculation portion 44 obtains data regarding the degree of metabolism based on the numerical value regarding the concentration of HHb. The present disclosure is not limited thereto. The concentration calculation portion 43 may calculate a numerical value regarding the concentration of O₂Hb, and the metabolism index calculation portion 44 may obtain data regarding the degree of metabolism based on the numerical value regarding the concentration of O₂Hb. Part (a) of FIG. 11 is a graph illustrating a time waveform of the temporal relative change amount of the concentration of HHb illustrated in Part (a) of FIG. 9. Part (b) of FIG. 11 is a graph illustrating a time waveform of the temporal relative change amount of the concentration of O₂Hb corresponding to Part (a) of FIG. 11. As will be apparent from comparison of Part (a) of FIG. 11 and Part (b) of FIG. 11, the time waveform of the numerical value regarding the concentration of O₂Hb has a shape obtained by vertically inverting the time waveform of the numerical value regarding the concentration of HHb. This is because the total concentration of hemoglobin is considered to be constant. Accordingly, similarly to a case where the numerical value regarding the concentration of HHb is used, even though the numerical value regarding the concentration of O₂Hb is used, data regarding the degree of metabolism can be obtained. For example, both data regarding the degree of metabolism that is obtained based on the numerical value regarding the concentration of HHb and data regarding the degree of metabolism that is obtained based on the numerical value regarding the concentration of O₂Hb may be obtained and may be averaged or the like, thereby increasing the accuracy of final data regarding the degree of metabolism. In this case, the concentration calculation portion 43 may obtain both the numerical value regarding the concentration of HHb and the numerical value regarding the concentration of O₂Hb may be obtained.

That is, in the concentration calculation processing 431, the concentration calculation portion 43 may obtain at least one parameter among the numerical value (third parameter) regarding the concentration of O₂Hb in blood from which the influence of the volume of the blood vessel in the optical path is excluded and the numerical value (fourth parameter) regarding the concentration of HHb in blood from which the influence of the volume of the blood vessel in the optical path is excluded, based on Δoxy-Hb and Δdeoxy-Hb calculated in the NIRS calculation portion 42. The numerical value regarding the concentration of O₂Hb in blood is, for example, the temporal relative change amount of the concentration of O₂Hb from a certain timing, typically, the time when measurement starts. Then, the metabolism index calculation portion 44 may obtain data (metabolism index) regarding the degree of metabolism based on a predetermined relationship between the numerical value regarding the concentration of O₂Hb, the numerical value regarding the concentration of HHb, or both the numerical values and the degree of metabolism in the metabolism index estimation processing 441. In this case, the concentration of HHb in the above description is appropriately substituted with the concentration of O₂Hb. In addition, Formula (27) described above is substituted with Formula (31) described below regarding C_{oxy,AC}(t), N_{oxy}(t), N_{deoxy}(t), and SpO₂.

$$[ Formula 31 ]$$

$$- {Sp O}_{2} \cdot {N}_{deoxy} ( t ) + ( 1 - {Sp O}_{2} ) \cdot {N}_{oxy} ( t ) \approx \frac{{V}_{DC}}{α} \cdot {C}_{oxy , DC} \qquad ( 31 )$$

Effects that are obtained by the metabolism measurement apparatus 1, the metabolism calculation method, and the metabolism calculation program of the present embodiment described above will be described along with the problems in the related art.

In recent years, apparatuses and methods for noninvasively measuring metabolism of a body have been developed. For example, an apparatus that measures a heat flow caused by metabolism of glucose in a body by a thermometer and estimates a blood glucose level from the heat flow is known. However, in such an apparatus, because a certain amount of time is required for heat conduction from a part of the body to the thermometer, there is a problem in that a lot of time is required for measurement. In addition, for example, an apparatus that estimates a degree of motion of a body based on a measured value of an accelerometer attached to a part of the body and calculates calorie consumption is known. An apparatus that measures amounts of oxygen included in exhalation and inhalation by a mask-shaped measurement apparatus covering a mouth and a nose and calculates an amount of oxygen consumption in a body by comparison of the amount of oxygen in exhalation and the amount of oxygen in inhalation is known. An apparatus that estimates a blood glucose level by absorbance measurement using an absorption peak of glucose in a mid-infrared wavelength region is known. An apparatus that measures a heat flow caused by metabolism of glucose in a body by a thermometer and estimates a blood glucose level from the heat flow is known.

However, each apparatus described above has problems and has not been widely spread. For example, in the apparatus that calculates calorie consumption based on the measured value of the accelerometer attached to a part of the body, there is difficulty in measuring basal metabolism not accompanied by movement. In the apparatus that calculates the amount of oxygen consumption in the body by comparison of the amount of oxygen in exhalation and the amount of oxygen in inhalation, there is difficulty in constantly wearing the measurement apparatus, and it is not suitable for measurement for a long time. In the apparatus that measures absorbance using the absorption peak of glucose, a concentration of glucose in a body tissue is very small, so that there is extreme difficulty in measuring the concentration of glucose with high accuracy. In the apparatus that measures the heat flow caused by the metabolism of glucose in the body, a lot of time is required for measurement of the heat flow by the thermometer.

The degree of metabolism of the body has a correlation with the concentration of O₂Hb and the concentration of HHb in blood. Accordingly, the degree of metabolism of the body can be measured noninvasively and in a short time by measuring any one or both of the concentration of O₂Hb and the concentration of HHb in blood. As a method for measuring the concentration of O₂Hb and the concentration of HHb in blood, near-infrared spectroscopy (NIRS) is known. In this method, each concentration of hemoglobin is measured in a body tissue in an optical path of measurement light. That is, each concentration of hemoglobin in blood itself is not obtained, and the obtained numerical value depends on each concentration of hemoglobin and the volume of the blood vessel in the optical path. Accordingly, the numerical value is affected by fluctuation of the volume of the blood vessel in the optical path, and in particular, fluctuation of an inner diameter of the blood vessel due to the beat of the heart, the metabolism of the body cannot be calculated using the numerical value as it is with high accuracy.

Therefore, in the present embodiment, first, in the NIRS calculation portion 42 and Step S15, Δoxy-Hb that is the temporal relative change amount depending on the concentration of O₂Hb in blood and the volume of the blood vessel in the optical path and Δdeoxy-Hb that is the temporal relative change amount depending on the concentration of HHb in blood and the volume of the blood vessel in the optical path are obtained in a similar manner to the NIRS of the related art. Then, in the concentration calculation portion 43 and Step S16, at least one numerical value between the numerical value regarding the concentration of O₂Hb in blood from which the influence of the volume of the blood vessel in the optical path is excluded and the numerical value regarding the concentration of HHb in blood from which the influence of the volume of the blood vessel in the optical path is excluded is obtained based on Δoxy-Hb and Δdeoxy-Hb. The present inventors have conducted studies and have found that the numerical value regarding each concentration of hemoglobin in blood from which the influence of the volume of the blood vessel in the optical path is excluded is obtained by calculation based on the numerical value obtained by the NIRS in this way. Data regarding the degree of metabolism can be obtained based on at least one numerical value in the metabolism index calculation portion 44 (Step S18). Therefore, according to the present embodiment, the degree of metabolism of the living body 50 can be measured noninvasively and with high accuracy, and can be measured in a short time to an extent equivalent to the NIRS of the related art. In addition, basal metabolism not accompanied by movement can be measured, the measurement apparatus is easily constantly worn, and measurement for a long time can be performed.

As described above, the concentration calculation portion 43 may obtain the numerical value regarding the concentration of O₂Hb and/or the concentration of HHb in blood assuming that the volume of the blood vessel in the optical path is constant. Similarly, in Step S16, the numerical value regarding the concentration of O₂Hb and/or the concentration of HHb in blood may be obtained assuming that the volume of the blood vessel in the optical path is constant. In a case where the volume of the blood vessel in the optical path can be assumed to be constant in a certain determined period (for example, several minutes to several months), or the like, the degree of metabolism of the living body 50 can be simply measured accordingly.

As in the present embodiment, the metabolism measurement apparatus 1 may include the blood vessel volume calculation portion 45 that obtains the numerical value regarding the fluctuation component of the volume of the blood vessel in the optical path from Δoxy-Hb and Δdeoxy-Hb. Then, the metabolism index calculation portion 44 may obtain data regarding the degree of metabolism based further on the numerical value regarding the fluctuation component of the volume of the blood vessel in the optical path obtained by the blood vessel volume calculation portion 45. Similarly, the metabolism calculation method and the metabolism calculation program may include, before Step S18, Step S17 of obtaining the numerical value regarding the fluctuation component of the volume of the blood vessel in the optical path from Δoxy-Hb and Δdeoxy-Hb. Then, in Step S18, data regarding the degree of metabolism may be obtained based further on the numerical value regarding the fluctuation component of the volume of the blood vessel in the optical path obtained in Step S17. In this way, the fluctuation component of the volume of the blood vessel in the optical path can be obtained by calculation based on the numerical value obtained by the NIRS. According to the studies of the present inventors, data regarding the degree of metabolism is obtained based further on the numerical value regarding the fluctuation component of the volume of the blood vessel in the optical path, so that reduction in measurement accuracy due to the fluctuation of the volume of the blood vessel in the optical path can be suppressed. The fluctuation of the volume of the blood vessel in the optical path can be caused by, for example, an increase or a decrease of a total amount of hemoglobin in a period from a certain measurement to next measurement, a change in the optical path due to attachment and detachment of the light measurement instrument 10, or a change in the volume of the blood vessel in the optical path due to a local change in a blood flow.

As in the present embodiment, the metabolism measurement apparatus 1 may include the blood vessel volume calculation portion 45 that extracts the steady components of Δoxy-Hb and Δdeoxy-Hb. Then, the metabolism index calculation portion 44 may obtain data regarding the degree of metabolism based further on the steady components of Δoxy-Hb and Δdeoxy-Hb obtained by the blood vessel volume calculation portion 45. Similarly, the metabolism calculation method and the metabolism calculation program may include, before Step S18, Step S17 of extracting the steady components of Δoxy-Hb and Δdeoxy-Hb. Then, in Step S18, data regarding the degree of metabolism may be obtained based further on the steady components of Δoxy-Hb and Δdeoxy-Hb obtained in Step S17. The steady components of Δoxy-Hb and Δdeoxy-Hb have a correlation with the volume of the blood vessel in the optical path. Accordingly, data regarding the degree of metabolism is obtained based further on the steady components of Δoxy-Hb and Δdeoxy-Hb, so that reduction in measurement accuracy due to the fluctuation of the volume of the blood vessel in the optical path can be suppressed.

As in the present embodiment, the concentration calculation portion 43 may obtain the numerical values regarding the concentration of O₂Hb and the concentration of HHb in blood from which the influence of the volume of the blood vessel in the optical path is excluded, based on Formulas (31) and (27), respectively. Similarly, in Step S16, the numerical values regarding the concentration of O₂Hb and the concentration of HHb in blood from which the influence of the volume of the blood vessel in the optical path is excluded may be obtained based on Formulas (31) and (27), respectively. With this, the degree of metabolism of the living body 50 can be calculated with higher accuracy.

As in the present embodiment, the metabolism index calculation portion 44 may obtain the blood glucose level of the living body 50 based on the pre-acquired relationship between the degree of metabolism and the blood glucose level. Similarly, in Step S18, the blood glucose level of the living body 50 may be obtained based on the pre-acquired relationship between the degree of metabolism and the blood glucose level. In this case, the blood glucose level can be measured noninvasively and in a short time. As described above, the pre-acquired relationship between the degree of metabolism and the blood glucose level may be appropriately corrected according to the change in the metabolic rate due to age and/or the change in the metabolic function due to the progress of a disease such as diabetes. Specifically, the relationship between the degree of metabolism and the blood glucose level may be corrected according to at least one parameter among the age, sex, height, and the degree of progress of the disease of the subject.

As in the present embodiment, the concentration calculation portion 43 may extract the quantity of predetermined feature from the numerical value regarding the concentration of O₂Hb and/or the concentration of HHb in blood, and the metabolism index calculation portion 44 may obtain data regarding the degree of metabolism based on the pre-acquired relationship between the quantity of predetermined feature and the degree of metabolism. Similarly, in Step S16, the quantity of predetermined feature may be extracted from the numerical value regarding the concentration of O₂Hb and/or the concentration of HHb in blood, and in Step S18, data regarding the degree of metabolism can be obtained based on the pre-acquired relationship between the quantity of predetermined feature and the degree of metabolism. In this case, data regarding the degree of metabolism can be obtained by simple calculation with high accuracy.

### Example

The present inventors have estimated metabolism indexes of three subjects using the metabolism measurement apparatus 1 according to the above-described embodiment, and have measured, as a reference example, the blood glucose levels of the same subjects using a household invasive blood glucose meter. Experimental conditions are as described below.
Measurement is performed using the metabolism measurement apparatus 1 and the invasive blood glucose meter at a predetermined time interval over 11 days after the subjects ingest a food including a lot of sugar
Ingesta: Coca-Cola (Registered Trademark) 1000 ml
Light measurement instrument 10 of metabolism measurement apparatus 1: pulse oximeter for earlobe
Measurement posture: supine position

Part (a) to Part (c) of FIG. 12 are graphs illustrating measurement results of the three subjects, respectively. In Part (a) to Part (c) of FIG. 12, the right vertical axis represents a blood glucose level (mg/dl), the left vertical axis represents a metabolism index (arbitrary unit), and the horizontal axis represents time (minute). In each drawing, a graph G41 indicates a result (see the right vertical axis) by the metabolism measurement apparatus 1, and a graph G42 indicates a result (see the left vertical axis) by the invasive blood glucose meter. Referring to Part (a) to Part (c) of FIG. 12, it is understood that a tendency of increase or decrease of the metabolism index estimated by the metabolism measurement apparatus 1 is quite approximate to a tendency of increase or decrease of an actual blood glucose level.

FIG. 13 is a graph illustrating a relationship between the metabolism index estimated by the metabolism measurement apparatus 1 and the actual blood glucose level. In FIG. 13, the vertical axis represents the metabolism index (arbitrary unit), and the horizontal axis represents the blood glucose level (mg/dl). A plot P1 in the drawing indicates a measurement result of a certain subject, a plot P2 in the drawing indicates a measurement result of another subject, and a plot P3 in the drawing indicates a measurement result of a further subject. A straight line R1 in the drawing is a regression line of the plots P1 to P3. A coefficient R² of determination of the straight line R1 is 0.4 and is large. In this way, it is understood that the metabolism index estimated by the metabolism measurement apparatus 1 and the actual blood glucose level have a substantially linear relationship.

In general, the blood glucose level and the metabolism index have a close relationship. The above-described experimental results indicate that the metabolism measurement apparatus 1 of the above-described embodiment can estimate the metabolism indexes of the subjects with high accuracy.

### Modification Example

In the above-described embodiment, the concentration calculation portion 43 extracts the quantity of predetermined feature (maximum value, time average value, peak-to-peak value, and time integration value) from the numerical value regarding the concentration of O₂Hb and/or the concentration of HHb in blood. In this case, the concentration calculation portion 43 may first execute filter processing on the numerical value regarding the concentration of O₂Hb and/or the concentration of HHb and may extract the quantity of predetermined feature from the numerical value after the filter processing. A filter that is used in the filter processing is at least one filter among a differentiation type filter, an integration type filter, a moving average filter, a low-pass filter, a high-pass filter, and a band-pass filter.

Here, combinations of various filters and the feature quantity will be examined. FIG. 14 is a diagram illustrating a graph (graph G51) that is a sinusoidal time waveform generated in a simulative manner for examination, on which white noise is superimposed, a graph (graph G52) obtained by applying an integration type filter to the graph G51, and a graph (graph G53) obtained by applying a differentiation type filter to the graph G51. In FIG. 14, the vertical axis represents a signal value (arbitrary unit), and the horizontal axis represents time (second). As illustrated in FIG. 14, when white noise is superimposed on the time waveform, noise is rather enhanced with the differentiation type filter. In contrast, it is understood that noise is effectively smoothed with the integration type filter.

Graphs G61 and G62 of Part (a) of FIG. 15 illustrate results of extracting maximum values of the graphs G51 and G52 of FIG. 14, respectively. Graphs G71 and G72 of Part (b) of FIG. 15 illustrate results of extracting peak-to-peak values of the graphs G51 and G52 of FIG. 14, respectively. Graphs G81 and G82 of Part (c) of FIG. 15 illustrate results of extracting time average values of the graphs G51 and G52 of FIG. 14, respectively. In Part (a) to Part (c) of FIG. 15, the vertical axis represents each value, and the horizontal axis represents time (second). A broken line in each drawing represents an ideal value. Referring to Part (a) to Part (c) of FIG. 15, when white noise is superimposed on an original time waveform, it is understood that any feature quantity converges to the ideal value with a small error by the application of the integration type filter, and estimation accuracy is improved. When the peak-to-peak value is set as the feature quantity, convergence is fast compared to when the time average value is set as the feature quantity, and when the time average value is set as the feature quantity, there is resistance to impulse-like noise compared to when the peak-to-peak value is set as the feature quantity.

FIG. 16 is a diagram illustrating a graph (graph G91) that is a sinusoidal time waveform generated in a simulative manner for examination, on which low-frequency undulations are superimposed, a graph (graph G92) obtained by applying an integration type filter to the graph G91, and a graph (graph G93) obtained by applying a differentiation type filter to the graph G91. In FIG. 16, the vertical axis represents a signal value (arbitrary unit), and the horizontal axis represents time (second). As illustrated in FIG. 16, when low-frequency undulations are superimposed on the time waveform, the value diverges with the integration type filter. In contrast, it is understood that low-frequency undulations are effectively removed with the differentiation type filter.

Graphs G101 to G103 of Part (a) of FIG. 17 illustrate results of extracting maximum values of the graphs G91 to G93 of FIG. 16, respectively. Graphs G111 to G113 of Part (b) of FIG. 17 illustrate results of extracting peak-to-peak values of the graphs G91 to G93 of FIG. 16, respectively. Graphs G121 to G123 of Part (c) of FIG. 17 illustrate results of extracting time average values of the graphs G91 to G93 of FIG. 16, respectively. In Part (a) to Part (c) of FIG. 17, the vertical axis represents each value, and the horizontal axis represents time (second). A broken line in each drawing represents an ideal value. Referring to Part (a) to Part (c) of FIG. 17, when low-frequency undulations are superimposed on an original time waveform, it is understood that any feature quantity converges to the ideal value fast with a small error by the application of the differentiation type filter, and estimation accuracy is improved. Also in this case, when the peak-to-peak value is set as the feature quantity, convergence is fast compared to when the time average value is set as the feature quantity. When the time average value is set as the feature quantity, there is resistance to impulse-like noise compared to when the peak-to-peak value is set as the feature quantity.

The metabolism measurement apparatus, the metabolism calculation method, and the metabolism calculation program according to the present invention are not limited to the above-described embodiment and the example, and various other modifications can be made. For example, although an example where the calculator 40 is incorporated in the main body unit 30 of a smart device or the like has been illustrated in the above-described embodiment, the calculator 40 may be provided in, for example, a cloud server or a personal computer separately from the main body unit 30. In this case, the calculator 40 may be connected to the main body unit 30 by a network such as wireless or the Internet. In the above-described embodiment, one calculation portion 40 includes the NIRS calculation portion 42, the concentration calculation portion 43, and the metabolism index calculation portion 44, but at least one calculation portion among the NIRS calculation portion 42, the concentration calculation portion 43, and the metabolism index calculation portion 44 may be provided separately from other calculation portions.

In the above-described embodiment, although a modified Beer-Lambert method (MBL method) has been described as the method for calculating Δoxy-Hb and Δdeoxy-Hb, other near-infrared spectroscopy such as space-resolved spectroscopy (SRS method) may be used.

In the above-described embodiment, data regarding the degree of metabolism is corrected using the numerical value regarding the fluctuation component of the volume of the blood vessel and the steady components of Δoxy-Hb and Δdeoxy-Hb. Data regarding the degree of metabolism may be corrected using at least one parameter among an air temperature, a body temperature, and a hematocrit value, in addition to or separately from the above-described values.

### REFERENCE SIGNS LIST

1 metabolism measurement apparatus, 10 light measurement instrument, 11 light source, 12 photodetector, 18 cable, 21 light source control unit, 22 sample-and-hold circuit, 23 A/D conversion circuit, 24 CPU, 25 display, 26 ROM, 27 RAM, 28 data bus, 29 controller, 30 main body unit, 31 input unit, 40 calculation portion, 41 measurement control portion, 42 NIRS calculation portion, 43 concentration calculation portion, 44 metabolism index calculation portion, 45 blood vessel volume calculation portion, 50 living body, 51 skin, 411 dark level measurement processing, 412 light output processing, 413 light detection processing, 421 buffer, 422 NIRS calculation processing, 423 SpO₂ calculation processing, 431 concentration calculation processing, 432 feature quantity extraction processing, 441 metabolism index estimation processing, 451 blood vessel volume estimation processing, B blood vessel, L1 measurement light.

1: A metabolism measurement apparatus that measures a degree of metabolism of a living body, the metabolism measurement apparatus comprising:
a light source configured to output measurement light to be input to the living body;
a light detector configured to detect the measurement light propagating through the living body and generates a detection signal according to an intensity of the measurement light; and
a processor configured to output data regarding the degree of metabolism based on the detection signal,
wherein the processor includes:
a first calculation portion configured to obtain, based on the detection signal, a first parameter that is a temporal relative change amount from a certain timing and depends on a concentration of oxygenated hemoglobin in blood and a volume of a blood vessel in an optical path and a second parameter that is a temporal relative change amount from a certain timing and depends on a concentration of deoxygenated hemoglobin in blood and the volume of the blood vessel in the optical path;
a second calculation portion configured to obtain, based on the first parameter and the second parameter, at least one parameter among a third parameter regarding a concentration of oxygenated hemoglobin in blood from which an influence of the volume of the blood vessel in the optical path is excluded and a fourth parameter regarding a concentration of deoxygenated hemoglobin in blood from which an influence of the volume of the blood vessel in the optical path is excluded; and
a third calculation portion configured to obtain the data regarding the degree of metabolism based on the at least one parameter.

2: The metabolism measurement apparatus according to claim 1,
wherein the second calculation portion obtains the at least one parameter assuming that the volume of the blood vessel in the optical path is constant.

3: The metabolism measurement apparatus according to claim 1, further comprising:
a fourth calculation portion configured to obtain a numerical value regarding a fluctuation component of the volume of the blood vessel in the optical path from the first parameter and the second parameter,
wherein the third calculation portion obtains the data regarding the degree of metabolism based further on the numerical value regarding the fluctuation component of the volume of the blood vessel in the optical path obtained by the fourth calculation portion.

4: The metabolism measurement apparatus according to claim 1, further comprising:
a fourth calculation portion configured to extract steady components of the first parameter and the second parameter,
wherein the third calculation portion obtains the data regarding the degree of metabolism based further on the steady components of the first parameter and the second parameter obtained by the fourth calculation portion.

5: The metabolism measurement apparatus according to claim 1,
wherein the second calculation portion obtains the third parameter based on Formula (1) in obtaining the third parameter, and obtain the fourth parameter based on Formula (2) in obtaining the fourth parameter (where SpO₂ is an oxygen saturation, N_{oxy}(t) is the first parameter, N_{deoxy}(t) is the second parameter, C_{oxy,AC} is the third parameter, C_{deoxy,AC} is the fourth parameter, V_{DC} is a steady component of the volume of the blood vessel in the optical path, and α is a constant).
$$[ Formula 1 ]$$
$$- {Sp O}_{2} \cdot {N}_{deoxy} ( t ) + ( 1 - {Sp O}_{2} ) \cdot {N}_{oxy} ( t ) \approx \frac{{V}_{DC}}{α} \cdot {C}_{oxy , AC} \qquad ( 1 )$$
$$[ Formula 2 ]$$
$${Sp O}_{2} \cdot {N}_{deoxy} ( t ) - ( 1 - {Sp O}_{2} ) \cdot {N}_{oxy} ( t ) \approx \frac{{V}_{DC}}{α} \cdot {C}_{deoxy , AC} . \qquad ( 2 )$$

6: The metabolism measurement apparatus according to claim 1,
wherein the third calculation portion further obtains a blood glucose level of the living body based on a pre-acquired relationship between the degree of metabolism and the blood glucose level.

7: The metabolism measurement apparatus according to claim 1,
wherein the second calculation portion extracts a quantity of predetermined feature from the at least one parameter, and
the third calculation portion obtains the data regarding the degree of metabolism based on a pre-acquired relationship between the quantity of predetermined feature and the degree of metabolism.

8: The metabolism measurement apparatus according to claim 7,
wherein the quantity of predetermined feature is at least one value selected from a group consisting of a maximum value, a time average value, a peak-to-peak value, and a time integration value, of the at least one parameter.

9: A metabolism calculation method that calculates a degree of metabolism of a living body, the metabolism calculation method comprising:
performing a first calculation of obtaining a first parameter that is a temporal relative change amount from a certain timing and depends on a concentration of oxygenated hemoglobin in blood and a volume of a blood vessel in an optical path and a second parameter that is a temporal relative change amount from a certain timing and depends on a concentration of deoxygenated hemoglobin in blood and the volume of the blood vessel in the optical path, in the living body;
performing a second calculation of obtaining, based on the first parameter and the second parameter, at least one parameter among a third parameter regarding a concentration of oxygenated hemoglobin in blood from which an influence of the volume of the blood vessel in the optical path is excluded and a fourth parameter regarding a concentration of deoxygenated hemoglobin in blood from which an influence of the volume of the blood vessel in the optical path is excluded; and
performing a third calculation of obtaining data regarding the degree of metabolism based on the at least one parameter.

10: The metabolism calculation method according to claim 9,
wherein, in the second calculation, the at least one parameter is obtained assuming that the volume of the blood vessel in the optical path is constant.

11: The metabolism calculation method according to claim 9, further comprising, before the third calculation:
performing a fourth calculation of obtaining a numerical value regarding a fluctuation component of the volume of the blood vessel in the optical path from the first parameter and the second parameter,
wherein, in the third calculation, the data regarding the degree of metabolism is obtained based further on the numerical value regarding the fluctuation component of the volume of the blood vessel in the optical path obtained in the fourth calculation.

12: The metabolism calculation method according to claim 9, further comprising, before the third calculation:
performing a fourth calculation of extracting steady components of the first parameter and the second parameter,
wherein, in the third calculation, the data regarding the degree of metabolism is obtained based further on the steady components of the first parameter and the second parameter obtained in the fourth calculation.

13: The metabolism calculation method according to claim 9,
wherein, in the second calculation, the third parameter is obtained based on Formula (3) in obtaining the third parameter and the fourth parameter is obtained based on Formula (4) in obtaining the fourth parameter (where SpO₂ is an oxygen saturation, N_{oxy}(t) is the first parameter, N_{deoxy}(t) is the second parameter, C_{oxy,AC} is the third parameter, C_{deoxy,AC} is the fourth parameter, V_{DC} is a steady component of the volume of the blood vessel in the optical path, and α is constant).
$$[ Formula 3 ]$$
$$- {Sp O}_{2} \cdot {N}_{deoxy} ( t ) + ( 1 - {Sp O}_{2} ) \cdot {N}_{oxy} ( t ) \approx \frac{{V}_{DC}}{α} \cdot {C}_{oxy , AC} \qquad ( 3 )$$
$$[ Formula 4 ]$$
$${Sp O}_{2} \cdot {N}_{deoxy} ( t ) - ( 1 - {Sp O}_{2} ) \cdot {N}_{oxy} ( t ) \approx \frac{{V}_{DC}}{α} \cdot {C}_{deoxy , AC} . \qquad ( 4 )$$

14: The metabolism calculation method according to claim 9,
wherein, in the third calculation, a blood glucose level of the living body is further obtained based on a pre-acquired relationship between the degree of metabolism and the blood glucose level.

15: The metabolism calculation method according to claim 9,
wherein, in the second calculation, a quantity of predetermined feature is extracted from the at least one parameter, and
in the third calculation, the data regarding the degree of metabolism is obtained based on a pre-acquired relationship between the quantity of predetermined feature and the degree of metabolism.

16: The metabolism calculation method according to claim 15,
wherein the quantity of predetermined feature is at least one value selected from a group consisting of a maximum value, a time average value, a peak-to-peak value, and a time integration value, of the at least one parameter.

17: A metabolism calculation program that calculates a degree of metabolism of a living body, the metabolism calculation program causing a computer to execute:
performing a first calculation of obtaining a first parameter that is a temporal relative change amount from a certain timing and depends on a concentration of oxygenated hemoglobin in blood and a volume of a blood vessel in an optical path and a second parameter that is a temporal relative change amount from a certain timing and depends on a concentration of deoxygenated hemoglobin in blood and the volume of the blood vessel in the optical path, in the living body;
performing a second calculation of obtaining, based on the first parameter and the second parameter, at least one parameter among a third parameter regarding a concentration of oxygenated hemoglobin in blood from which an influence of the volume of the blood vessel in the optical path is excluded and a fourth parameter regarding a concentration of deoxygenated hemoglobin in blood from which an influence of the volume of the blood vessel in the optical path is excluded; and
performing a third calculation of obtaining data regarding the degree of metabolism based on the at least one parameter.

18: The metabolism calculation program according to claim 17,
wherein, in the second calculation, the at least one parameter is obtained assuming that the volume of the blood vessel in the optical path is constant.

19: The metabolism calculation program according to claim 17, causing the computer to further execute, before the third calculation:
performing a fourth calculation of obtaining a numerical value regarding a fluctuation component of the volume of the blood vessel in the optical path from the first parameter and the second parameter,
wherein, in the third calculation, the data regarding the degree of metabolism is obtained based further on the numerical value regarding the fluctuation component of the volume of the blood vessel in the optical path obtained in the fourth calculation.

20: The metabolism calculation program according to claim 17, causing the computer to further execute, before the third calculation:
performing a fourth calculation of extracting steady components of the first parameter and the second parameter,
wherein, in the third calculation, the data regarding the degree of metabolism is obtained based further on the steady components of the first parameter and the second parameter obtained in the fourth calculation.
